SEQUENCE LISTING (62 sequences)

SEQ ID NO: 1 - DNA, 358 bp, synthetic construct
  source 1..358: mol_type = other DNA
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 2 - DNA, 612 bp, synthetic construct
  source 1..612: mol_type = other DNA
cactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggcgcgccgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatat

SEQ ID NO: 3 - DNA, 321 bp, synthetic construct
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 4 - DNA, 397 bp, synthetic construct
  source 1..397: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 5 - DNA, 683 bp, synthetic construct
  source 1..683: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtaccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 6 - DNA, 1121 bp, synthetic construct
  source 1..1121: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggaccccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 7 - protein, 7 aa, synthetic construct
RGDLGLS

SEQ ID NO: 8 - protein, 7 aa, synthetic construct
LRGDGLS

SEQ ID NO: 9 - protein, 7 aa, synthetic construct
LGRGDLS

SEQ ID NO: 10 - protein, 7 aa, synthetic construct
LGLRGDS

SEQ ID NO: 11 - protein, 7 aa, synthetic construct
LGLSRGD

SEQ ID NO: 12 - protein, 7 aa, synthetic construct
RGDMSRE

SEQ ID NO: 13 - protein, 9 aa, synthetic construct
CDCRGDCFC

SEQ ID NO: 14 - protein, 7 aa, synthetic construct
RGDAVGV

SEQ ID NO: 15 - protein, 7 aa, synthetic construct
PRGDLAP

SEQ ID NO: 16 - protein, 7 aa, synthetic construct
RGDVAAK

SEQ ID NO: 17 - protein, 7 aa, synthetic construct
RGDMINT

SEQ ID NO: 18 - protein, 7 aa, synthetic construct
RGDLNDS

SEQ ID NO: 19 - protein, 7 aa, synthetic construct
RGDTMNY

SEQ ID NO: 20 - protein, 7 aa, synthetic construct
RGDLTTP

SEQ ID NO: 21 - protein, 7 aa, synthetic construct
RGDLNQY

SEQ ID NO: 22 - protein, 7 aa, synthetic construct
RGDLSTP

SEQ ID NO: 23 - protein, 7 aa, synthetic construct
RGDQLYH

SEQ ID NO: 24 - protein, 7 aa, synthetic construct
RGDTMSK

SEQ ID NO: 25 - protein, 7 aa, synthetic construct
RGDATEL

SEQ ID NO: 26 - protein, 10 aa, synthetic construct
GPGRGDQTTL

SEQ ID NO: 27 - protein, 10 aa, synthetic construct
AEGRGDQYTR

SEQ ID NO: 28 - protein, 10 aa, synthetic construct
ATGRGDLGQA

SEQ ID NO: 29 - protein, 10 aa, synthetic construct
AVARGDQGLI

SEQ ID NO: 30 - protein, 10 aa, synthetic construct
NISRGDQGYQ

SEQ ID NO: 31 - protein, 10 aa, synthetic construct
APARGDQGSQ

SEQ ID NO: 32 - protein, 10 aa, synthetic construct
AVSRGDRMEF

SEQ ID NO: 33 - protein, 10 aa, synthetic construct
SPSRGDQGRT

SEQ ID NO: 34 - protein, 7 aa, synthetic construct
RGDYVGL

SEQ ID NO: 35 - protein, 7 aa, synthetic construct
RGDYSGL

SEQ ID NO: 36 - protein, 7 aa, synthetic construct
RGDYSSV

SEQ ID NO: 37 - protein, 7 aa, synthetic construct
RGDYREL

SEQ ID NO: 38 - protein, 7 aa, synthetic construct
RGDHGVL

SEQ ID NO: 39 - protein, 7 aa, synthetic construct
RGDHASW

SEQ ID NO: 40 - protein, 10 aa, synthetic construct
SNSRGDYNSL

SEQ ID NO: 41 - protein, 9 aa, synthetic construct
STVRGDYTS

SEQ ID NO: 42 - protein, 10 aa, synthetic construct
QERRGDYTSM

SEQ ID NO: 43 - protein, 10 aa, synthetic construct
ASTRGDHGVL

SEQ ID NO: 44 - protein, 10 aa, synthetic construct
ENRRGDFNNT

SEQ ID NO: 45 - protein, 4 aa, synthetic construct
GQSG

SEQ ID NO: 46 - protein, 4 aa, synthetic construct
AQAA

SEQ ID NO: 47 - protein, 15 aa, synthetic construct
GQSGRGDLGLSAQAA

SEQ ID NO: 48 - protein, 738 aa, Adeno-associated dependoparvovirus A
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, Adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 50 - protein, 738 aa, Adeno-associated dependoparvovirus A
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 51 - protein, 737 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 770 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLEFAVNTEGVYSEPRPIGTRYLTRNL

SEQ ID NO: 53 - protein, 749 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAARGDLGLSSGAAPIVGAVNSQGALPGMVWQNRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 54 - protein, 746 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQGQSGRGDLGLSAQAAIVGAVNSQGALPGMVWQNRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 55 - protein, 746 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDTESVPDPQPIGEPPAAPAAVGPTTMASGGGAPMADNNEGADGVGNASGNWHCDSTWLGDRVITTSTRTWALPTYNNHLYKQISNGTSGGATNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 56 - protein, 744 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGNASGNWHCDSQWLGDRVITTSTRTWALPTYNNHLYKQISSASTGATNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 57 - DNA, 22 bp, synthetic construct
  source 1..22: mol_type = other DNA
agatacgccggacattggactg

SEQ ID NO: 58 - DNA, 20 bp, synthetic construct
  source 1..20: mol_type = other DNA
gttcaatctgctgggcgtgc

SEQ ID NO: 59 - DNA, 19 bp, synthetic construct
  source 1..19: mol_type = other DNA
gtgtggtcctcttgggagc

SEQ ID NO: 60 - DNA, 21 bp, synthetic construct
  source 1..21: mol_type = other DNA
aaaacgagcagtgacgtgagc

SEQ ID NO: 61 - DNA, 20 bp, synthetic construct
  source 1..20: mol_type = other DNA
ttcagtcatgctgctagcgc

SEQ ID NO: 62 - DNA, 24 bp, synthetic construct
  source 1..24: mol_type = other DNA
tgcacggaagcgtctcgtctcagt

(19) United States
(12) Patent Application Publication
(10) Pub. No.: US 2026/0250712 A1
(43) Pub. Date: Aug. 27, 2026

(54) Induction of Immune Tolerance by AAV Vector Comprising the Combination of a Liver Detargeted Capsid and a Tandem Liver-Muscle Specific Promoter

(71) Applicants: GENETHON, Evry-Courcouronnes (FR); UNIVERSITE D'EVRY VAL D'ESSONNE, Evry-Courcouronnes (FR); INSTITUT NATIONAL DE LA SANTÉ ET DE LA RECHERCHE MÉDICALE, Paris (FR)

(72) Inventors: Giuseppe RONZITTI, FONTAINEBLEAU (FR); Pauline VIDAL, BOUSSY SAINT ANTOINE (FR)

(73) Assignees: GENETHON, Evry-Courcouronnes (FR); UNIVERSITE D'EVRY VAL D'ESSONNE, Evry-Courcouronnes (FR); INSTITUT NATIONAL DE LA SANTÉ ET DE LA RECHERCHE MÉDICALE, Paris (FR)

(21) Appl. No.: 19/163,537

(22) PCT Filed: Mar. 8, 2024
(86) PCT No.: PCT/EP2024/056304

§ 371 Date: Sep. 9, 2025

(30) Foreign Application Priority Data
Mar. 10, 2023 (EP) 23305332.1

(51) Int. Cl. C12N 15/86; A61K 48/00; C07K 14/005

(52) U.S. Cl. CPC C12N 15/86; A61K 48/0041; A61K 48/0058; C07K 14/005; C12N 2750/14122; C12N 2750/14143; C12N 2750/14145; C12N 2800/22; C12N 2830/008; C12N 2830/50

(57) ABSTRACT

The invention relates to an AAV vector comprising the combination of a tandem liver-muscle selective promoter and a liver detargeted AAV capsid protein modified with a muscle-targeting peptide and its use in gene therapy of muscle diseases, in particular genetic neuromuscular diseases.

### FIELD OF THE INVENTION

The invention relates to an AAV vector comprising the combination of a tandem liver-muscle selective promoter and a liver-detargeted AAV capsid protein modified with a muscle-targeting peptide and its use in gene therapy of muscle diseases, in particular genetic neuromuscular diseases.

### BACKGROUND OF THE INVENTION

Recombinant Adeno-Associated Virus (rAAV or AAV) vectors are widely used for in vivo gene transfer and clinical trials using AAV vectors are currently taking place for the treatment of a number of diseases.

AAV is a non-pathogenic virus belonging to the genus Dependoparvovirus within the family Parvoviridae. AAV is a non-enveloped virus composed of a capsid of about 25 nm in diameter and a single-stranded DNA genome of 4.7 kb. The genome carries two genes, rep and cap, flanked by two palindromic regions named Inverted terminal Repeats (ITR) that serve as the viral origins of replication and the packaging signal. The cap gene codes for three structural proteins VP1, VP2 and VP3 that compose the icosahedral AAV capsid through alternative splicing and translation from different start codons. VP1, VP2 and VP3 share the same C-terminal end which is all of VP3. Using AAV2 has a reference, VP1 has a 735 amino acid sequence (GenBank accession number YP_680426.1 accessed on 13 Aug. 2018); VP2 (598 amino acids) starts at the Threonine 138 (T138) and VP3 (533 amino acids) starts at the methionine 203 (M203). The rep gene encodes four proteins required for viral replication Rep78, Rep68, Rep52 and Rep40. Recombinant AAV vectors encapsidate an ITR-flanked rAAV genome in which a therapeutic gene expression cassette replaces the AAV protein coding-sequences.

The immune response to the transgene represents a limitation of current AAV vectors for gene therapy of muscle diseases.

It was shown previously that a strong expression of the transgene in the liver mediated by AAV, induced an immunological tolerance to transgenes expressed in muscle (Franco, L. M., et al., Mol Ther, 2005. 12 (5): p. 876-84; Zhang, P., et al., Hum Gene Ther, 2012. 23 (5): p. 460-72; Poupiot, J., et al., Molecular Therapy-Methods & Clinical Development, 2019. 15: p. 83-100; Bartolo, L., et al., JCI insight, 2019. 4 (11)).

Consistently, it was demonstrated that combined expression of human acid α-glucosidase (hGAA) in liver and muscle with a liver-muscle tandem promoter (LiMP) carried by AAV vector (AAV9-LiMP), resulted in very low humoral immunity to hGAA in Gaa^{-/-} mice, compared to a specific-muscle promoter or ubiquitous promoter (Colella, P., et al., Mol Ther Methods Clin Dev, 2019. 12: p. 85-101; WO 2019/154939). High expression of the transgene in liver and muscle is beneficial for gene therapy of some diseases such as Pompe's disease.

However, high expression of the transgene in the liver is not desired for the treatment of a large number of muscle diseases and may induce liver toxicity.

Therefore, there is a need for improved AAV vectors for gene therapy of muscle diseases.

### SUMMARY OF THE INVENTION

To study the contribution of muscle-specific expression of hGAA transgene in AAV gene therapy of Pompe's disease, the inventors have modified the previous AAV9-LiMP vector to replace the liver-tropic AAV9 capsid with a liver-detargeted capsid grafted with a muscle-targeting peptide comprising a RGD-motif (AAV-MT), previously described in WO 2020/200499. Surprisingly, despite its extensive liver-detargeting, this new AAV vector (AAV-MT-LiMP) had limited immune response to the transgene (FIG. 6E). Complete liver detargeting by AAV-MT led to increased humoral immune response to hGAA, while very low liver targeting, achieved with higher doses of the same capsid, was sufficient to reduce this immune response. These results support the importance of the residual liver expression to control the immune response toward a muscle-specific expression of an immunogenic transgene. Furthermore, specific muscle targeting achieved by AAV-MT-LiMP, demonstrated a clear dose-advantage in muscle correction when compared to AAV9-LiMP (FIGS. 4B-C-D-E). Altogether, these results demonstrate that this new AAV vector combines advantageously an increased muscle transduction efficiency and a reduced immune response to the transgene together with an extensive liver-detargeting which avoids liver toxicity due to liver overload.

The invention relates to an adeno-associated virus (AAV) vector comprising:
a transgene of interest operably linked to a tandem promoter comprising a muscle-selective promoter fused to a liver-selective promoter; and
a peptide-modified AAV capsid protein which is detargeted from the liver and comprises the insertion of a muscle-targeting peptide.

In some embodiments, the AAV vector according to the invention induces immune tolerance to the transgene of interest.

In some embodiments of the AAV vector according to the invention, the muscle-selective promoter is selected from the group consisting of: a spC5.12 promoter, the MHCK7 promoter, the E-syn promoter, a muscle creatine kinase myosin light chain (MLC) promoter, a myosin heavy chain (MHC) promoter, a desmin promoter, a cardiac troponin C promoter, a troponin I promoter, a myoD gene family promoter, an alpha actin promoter, an beta actin promoter, an gamma actin promoter, a muscle-selective promoter residing within intron 1 of the ocular form of Pitx3 and a CK6 promoter; preferably spC5.12 promoter of SEQ ID NO: 1.

In some embodiments of the AAV vector according to the invention, the liver-selective promoter is selected from the group consisting of: an alpha-1 antitrypsin promoter (hAAT), a combination of the ApoE enhancer and an hAAT promoter, a transthyretin promoter, an albumin promoter, a thyroxine-binding globulin (TBG) promoter and the LSP promoter; preferably comprising the combination of the ApoE enhancer (ApoE) of SEQ ID NO: 3 and hAAT promoter of SEQ ID NO: 4.

In some embodiments of the AAV vector according to the invention, the tandem promoter further comprises a muscle-selective enhancer and/or a liver-selective enhancer. In some particular embodiments, the tandem promoter comprises the combination of the ApoE enhancer with a liver-selective promoter as disclosed herein.

In some particular embodiments of the AAV vector according to the invention, the tandem promoter comprises the combination of: (i) the ApoE enhancer and hAAT promoter and (ii) the spC5.12 promoter; in particular comprising SEQ ID NO: 5 or 6; more particularly comprising SEQ ID NO: 6.

In some embodiments of the AAV vector according to the invention, the gene of interest is a therapeutic gene.

In some embodiments of the AAV vector according to the invention, the peptide-modified AAV capsid protein comprises a muscle-targeting peptide comprising a RGD motif; preferably comprising a sequence selected from the group consisting of SEQ ID NO: 7 to 44 and 47; preferably SEQ ID NO: 7.

In some embodiments of the AAV vector according to the invention, the peptide-modified AAV capsid protein is from AAV serotype selected from the group consisting of: AAV8, AAV9, AAVrh74, variants and hybrids thereof; preferably hybrid serotype AAV9.rh74; and AAV8, AAV9 or AAVrh74 hybrid serotypes comprising variable region(s) from AAV13 or hybrid AAV2/13.

In some embodiments of the AAV vector according to the invention, the muscle-targeting peptide is inserted in the variable region VIII of AAV capsid protein; preferably in a position of AAV capsid protein sequence selected from the group consisting of: position 585 or 590 in AAV8, position 588 or 589 in AAV9, position 589 in AAV9.rh74 and between positions 586 and 593 in AAV9.rh74.

In some embodiments of the AAV vector according to the invention, the modified AAV capsid comprises a sequence having at least 95% identity with any one of SEQ ID NO: 52 to 56 which comprises said peptide P1; preferably comprising SEQ ID NO: 54.

In some preferred embodiments of the AAV vector according to the invention, the tandem promoter comprises or consists of SEQ ID NO: 6 and the peptide-modified AAV capsid comprises or consists of SEQ ID NO: 54.

The invention further relates to a pharmaceutical composition comprising a therapeutically effective amount of AAV vector according to the present disclosure, or cell stably transduced by said AAV vector. The invention also relates to the pharmaceutical composition according to the present disclosure for use as a medicament in gene therapy; in particular for use in the treatment of muscle diseases; preferably selected from the group consisting of: Duchenne muscular dystrophy, Limb-girdle muscular dystrophies, Spinal muscular atrophy, Myotubular myopathy, Pompe disease and Glycogen storage disease III. In some embodiments, the pharmaceutical composition for use according to the invention targets a gene selected from the group comprising: DMD, CAPN3, DYSF, FKRP, DNAJB6, ANO5, SGCA, SGCB, SGCG, SMN1, ASAH, MTM1, GAA and AGL.

### DETAILED DESCRIPTION OF THE INVENTION

The invention relates to an adeno-associated virus (AAV) vector comprising: a transgene of interest operably linked to a tandem promoter comprising a muscle-selective promoter fused to a liver-selective promoter; and a peptide-modified AAV capsid protein which is detargeted from the liver and comprises the insertion of a muscle-targeting peptide. The invention encompasses the use of the AAV vector for gene therapy, in particular for treating muscle diseases such as genetic neuromuscular diseases.

As used herein, “AAV vector” refers to an AAV vector particle composed of genetic material made from DNA (i.e., AAV vector genome) surrounded by a protein coat, called the capsid (i.e., AAV capsid). AAV vector according to the invention refers to a recombinant AAV (rAAV) vector produced by standard recombinant DNA technology techniques that are known in the art.

As used herein, the term “target tissue or organ” refers to an individual tissue or organ or plurality of tissues or organs where expression of the transgene of interest by the AAV vector will be useful for a desired application. In particular, the target tissue(s) or organ(s) are targeted for gene therapy, i.e., for treating diseases by administration of the AAV vector according to the invention. The AAV vector according to the invention targets muscle tissue (i.e., muscles) and liver tissue (i.e., liver). Residual expression is sought in the liver to induce immune tolerance to the transgene of interest, while strong expression is sought in muscle to induce a therapeutic effect, in particular for treating muscle diseases. Induction of immune tolerance limits the immune response against the transgene and thereby increase the therapeutic efficacy of the AAV vector according to the invention.

As used herein, the term “muscle” refers to cardiac muscle (i.e. heart) and skeletal muscle. The term “muscle cells” refers to myocytes, myotubes, myoblasts, and/or satellite cells. As used herein “liver cells” include hepatocytes (liver parenchymal cells) that constitute about 80% of liver cells.

As used herein, ‘disease” or “disorder” refer to a disease that can be treated by gene therapy using the AAV vector according to the invention. A disease includes in particular, a disease associated with a gene mutation (genetic disease) and therefore eligible to AAV gene therapy.

In the context of the present invention, a “tolerogenic tissue”, is a tissue, such as liver, from which immune tolerance against a transgene may be achieved when said transgene is expressed from said tissue.

The term “immune tolerance” refers to a state of unresponsiveness to a specific antigen or group of antigens to which a subject would normally be responsive. Alternatively, immune tolerance can be defined as a state in which the immune system actively mediates suppression of immune responses to an antigen, for example via regulatory T cells. In the context of the present invention, the “antigen” or “group of antigens” against which immune tolerance is sought to be achieved is the transgene of interest. Immune tolerance is in particular induced to the transgene expressed in a target cell tissue or organ of interest for therapy, more particularly to the transgene of interest expressed in muscles.

The examples show that after systemic administration of a reduced dose of AAV vector which detargets the capsid from the liver, which means that results in no or almost no transgene expression in the liver, the levels of circulating anti-transgene IgG increased overtime (see FIGS. 6A-B-C and FIG. 6E; dose of 1×10¹² vector genomes (vg) of AAV-MT per kg for mice). In contrast, at the higher dose of AAV vector which induces residual expression of the transgene in the liver, anti-transgene IgG decreased dramatically (see FIGS. 6A-B-C and FIG. 6E; dose of 3×10¹² vector genomes (vg) of AAV-MT per kg for mice).

Therefore, induction of immune tolerance to the transgene by the AAV vector according to the invention may be determined by measuring the level of circulating IgG against the transgene after systematic administration of two doses of AAV vector, a low dose (liver detargeting dose) used for comparison and a higher dose (low liver-targeting dose), at different time points (for example 1 month and 3 months). IgG anti-transgene levels may be measured by standard ELISA assay as disclosed in the examples. The higher dose of AAV vector reduces the immune response to the transgene over time indicating the induction of an immune tolerance to the transgene.

In the following description, the amino acid residues are designated by the standard one letter amino acid code. In the present description, an insertion at or into a given position of AAV capsid protein sequence refers to an insertion after the amino acid residue at that position in VP1 amino acid sequence.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

### Peptide-Modified AAV Capsid

The AAV vector according to the invention comprises a peptide-modified AAV capsid protein which is detargeted from the liver by the insertion of a muscle-targeting peptide.

As used herein, a “muscle-targeting peptide” refers to a peptide which binds selectively to muscle cells and directs or targets capsid-modified AAV vectors carrying the peptide preferably to muscle cells and tissue in vivo as compared to other cells or tissues including liver cells and liver tissue. As a result, the peptide-modified AAV capsid has an increased transduction efficiency in muscle (muscle on-targeting).

The muscle on-targeting and liver-detargeting of the modified AAV capsid protein according to the invention may be determined by measuring the ability of AAV vector particles comprising the modified AAV capsid protein to transduce muscle cell, tissue or organ and liver cell, tissue or organ in vivo using standard assays that are well-known in the art such as those disclosed in the examples of the present application. For example, muscle and liver transduction levels may be determined by systemic administration of AAV vector particles carrying the modified AAV capsid protein in animal models such as mouse models that are well known in the art and disclosed in the examples of the present application. AAV vectors comprising unmodified AAV capsid protein are used for comparison.

AAV vector transduction may be determined in vitro or in vivo by measuring vector genome copy number or transgene expression. Vector genome copy number per diploid genome may be measured by standard assays that are well known in the art such as real-time PCR assay. Transgene expression is advantageously measured using a reporter gene such as luciferase or fluorescent protein (GFP or others) by standard assays that are well known in the art such as in vivo or in vitro quantitative bioluminescence or fluorescence assays in vivo or in vitro. Transgene expression may also be advantageously measured using a gene encoding a secreted protein that is expressed in the liver and secreted from the liver into the bloodstream. The level of secreted protein in the serum may be measured by standard assays that are well known in the art such as ELISA.

A muscle on-targeting refers to an increased level (higher level or elevated level) of transduction in muscle, in particular to a transgene expression level that is increased in at least one muscle cell, tissue or organ, compared to unmodified AAV capsid protein or a vector copy number that is increased in at least one muscle cell, tissue or organ, compared to unmodified AAV capsid protein. The muscle on-targeting properties of the peptide-modified AAV capsid according to the invention are shown in the examples of the present application (see for examples FIGS. 4B-4C-4D; 5A-5C).

As used herein, an AAV capsid protein which is detargeted from the liver, refers to an AAV capsid which induces no or almost no transduction in liver after systemic administration of a reduced dose (low dose or lower dose) of AAV vector as described herein, in particular to no or almost no transgene expression or vector copy number in liver cell, tissue or organ compared to unmodified AAV capsid protein (see for examples FIGS. 6A-6B-6C). In contrast to the liver, muscles are transduced very efficiently with the low dose of AAV vector comprising a liver-detargeted capsid (FIGS. 4C-D). In contrast to the low dose of AAV vector, a higher dose of AAV vector as described herein induces a residual expression of the transgene in the liver (see for example FIGS. 2D, 3B, 6A, 6B, 6C).

Liver detargeting may be achieved using an AAV capsid which is detargeted from the liver such as AAV9.rh74 (as disclosed in WO2019/193119, in particular the sequence SEQ ID NO: 51 as described herein). Alternatively or additionally, liver detargeting may be achieved by insertion of the muscle-targeting peptide at a site of the capsid that is involved in liver targeting such as the HSPG binding site for rodents; liver-detargeting can be achieved for example by insertion of the peptide into the Variable Region VIII.

The peptide-modified (or modified) AAV capsid protein according to the invention is a functional AAV capsid which is able to form recombinant AAV vector particles which transduce a cell, tissue or organ, in particular a cell tissue or organ of interest (target cell, tissue or organ) and express a transgene in said cell, tissue or organ, in particular target cell tissue or organ. A modified AAV capsid protein according to the invention is a recombinant protein.

In some embodiments, the muscle-targeting peptide comprises an RGD motif which is known to bind several different cell-surface integrins; AAV capsid modified with RGD containing peptide have been reported to improve gene delivery in muscle and induce liver detargeting following systemic administration (WO 2020/200499; WO 2019/207132; WO2022/053630; Weinmann et al., Nature communications, 2020, 11, 5432; Tabebordbar et al., Cell, 2021, 184, 4919-4938). RGD-containing peptides that can be inserted in the peptide-modified AAV capsid according to the invention include with no limitations: P1 (RGDLGLS), LRGDGLS, LGRGDLS, LGLRGDS, LGLSRGD, RGDMSRE, P2 (CDCRGDCFC), P3 (RGDAVGV), Kera2 (PRGDLAP), RGDVAAK, RGDMINT, RGDLNDS, RGDTMNY, MyoAAV 1A (RGDLTTP), MyoAAV 1B (RGDLNQY), MyoAAV 1C (RGDLSTP), MyoAAV 1D (RGDQLYH), MyoAAV 1E (RGDTMSK), MyoAAV 1F (RGDATEL), MyoAAV 2A (GPGRGDQTTL), MyoAAV 2B (AEGRGDQYTR), MyoAAV 2C (ATGRGDLGQA), MyoAAV 2D (AVARGDQGLI), MyoAAV 2E (NISRGDQGYQ), MyoAAV 2F (APARGDQGSQ), MyoAAV 2G (AVSRGDRMEF), MyoAAV 2H (SPSRGDQGRT), MyoAAV 3A (RGDYVGL), MyoAAV 3B (RGDYSGL), MyoAAV 3C (RGDYSSV), MyoAAV 3D (RGDYREL), MyoAAV 3E (RGDHGVL), MyoAAV 3F (RGDHASW), MyoAAV 4A (SNSRGDYNSL), MyoAAV 4B (STVRGDYTS), MyoAAV MyoAAV 4C (QERRGDYTSM), MyoAAV 4D (ASTRGDHGVL), and MyoAAV 4E (ENRRGDFNNT), corresponding to SEQ ID NO: 7 to 44. In some particular embodiments, the RGD-containing peptide comprises or consists of a sequence selected from the group consisting of: SEQ ID NO: 7 to 44; preferably SEQ ID NO: 7 (RGDLGLS or P1).

The muscle-targeting peptide consists generally of a sequence of up to 30 amino acids. The targeting peptide may consist of 9, 10, 11, 12, 13, 14, 15, 16, 17, 18, 19, 20, 21, 22, 23, 24, 25, 26, 27, 28, 29, 30 amino acids. In some embodiments, the muscle-targeting peptide consists of a sequence of up to 25, 20 or 15 amino acids. Preferably, the targeting peptide consists of a sequence of 12, 13, 14 or 15 amino acids. In addition, the muscle-targeting peptide may comprise flanking sequence(s) of up to five amino acids (1, 2, 3, 4 or 5) at its N-ter and/or C-ter end(s), wherein the flanking sequences may be the same or different. Examples of flanking sequences include GQSG and AQAA, respectively at the N- and C-terminal end of the muscle-targeting peptide.

A preferred peptide is RGDLGLS flanked by GQSG (SEQ ID NO: 45) and AQAA (SEQ ID NO: 46), respectively at its N- and C-terminal end, corresponding to GQSGRGDLGLSAQAA (SEQ ID NO: 47).

The muscle-targeting peptide is inserted into a site exposed on the capsid surface. Site exposed on the AAV capsid surface are well-known in the art and include in particular the variable regions (VRs or hypervariable regions, HVRs) which form loops at the top of the protrusions, such as VR-IV, -V and -VIII (Review in Büning et al., Molecular Therapy: Methods & Clinical Development, 2019, 12, 248-). VR-IV corresponds to Y445 to A476 (broad definition) or Q451 to L462 (narrow definition); VR-V corresponds to C485 to G515 (broad definition) or R490 to T509 (narrow definition); VR-VIII corresponds to I581 to L604 (broad definition) or L586 to I595 (narrow definition) according to the numbering in AAV8 capsid protein sequence. The peptide insertion site is advantageously at a site of the common VP3 region suitable exposed on the AAV capsid surface such as for example position 587, 588, 589, 453, 520 (combined with 584), 584 and 585, according to the numbering in AAV2 capsid protein sequence. The peptide insertion sites are indicated by reference to AAV2 or AAV8 capsid amino acid sequence. After sequence alignment of any other AAV capsid sequence with AAV2 or AAV8 capsid sequence using standard protein sequence alignment programs that are well-known in the art, such as for example BLAST, FASTA, CLUSTALW, and the like, a person skilled in the art can easily obtained the corresponding positions of the peptide insertion sites in other AAV capsid sequences.

In some particular embodiments, the muscle-targeting peptide is inserted into the variable region VIII. Preferred insertion sites for AAV serotypes include position 590 in AAV1; positions 587 or 588 in AAV2; position 586 in AAV3 or AAV4; position 575 in AAV5; position 585 in AAV6; positions 585 or 590 in AAV8; positions 588 or 589 in AAV9; position 589 in AAV9.rh74; between positions 586 (Q586) and 593 (1593) in AAV9.rh74.

The modified AAV capsid protein may comprise one or more muscle-targeting peptide insertions at different sites of the AAV capsid protein, wherein the inserted peptides may have the same sequence or different sequences.

The muscle-targeting peptide may be inserted between 2 consecutive amino acids of the AAV capsid protein sequence (no deletion) or may replace some or all of the residue(s) from the insertion site (deletion).

The modified AAV capsid protein may be derived from any natural or artificial AAV capsid serotype including hybrid serotypes and variant serotypes. Numerous AAV serotypes including AAV1 to 13, AAVrh10, AAVrh39, AAVrh43, AAVrh74, have been isolated in human and non-human primates. AAV2 variant serotypes and AAV2/13 hybrid capsids have been isolated in human liver (La Bella et al., Gut, 2020, 69, 737-747.doi: 10.1136/gutjnk-2019-318281; WO 2020/216861). Other AAV serotypes have been isolated in non-primate species, such as porcine, bovine, avian and caprine. Porcine AAV includes in particular AAVpo1, po2.1, po4 to 6. Various AAV capsid variants, also named “synthetic AAV serotypes”, “new AAV serotypes” or “AAV hybrid serotypes” have been engineered, in particular by rational design, directed gene evolution and in silico discovery. Non limiting examples of these new serotypes include: recombinant AAV2-derived serotypes DJ, DJ8 and PHP such as PHP.B and PHP.EB which are hybrid capsids from 8 AAV serotypes (AAV2, 4, 5, 8, 9, avian, bovine and goat), AAV-Anc80, AAV2i8, AAV-LK03, AAV2 comprising an engineered capsid with Y44+500+730F+T491V changes, disclosed in Ling et al., 2016 Jul. 18, Hum Gene Ther Methods.), AAV3 variants (such as the AAV3-ST variant comprising an engineered AAV3 capsid with two amino acid changes, S663V+T492V, disclosed in Vercauteren et al., 2016, Mol. Ther. Vol. 24 (6), p. 1042), AAV- 3B variants, AAV6 variants (such as the AAV6 variant comprising the triply mutated AAV6 capsid Y731F/Y705F/T492V form disclosed in Rosario et al., 2016, Mol Ther Methods Clin Dev. 3, p. 16026), AAV 2G9, AAVcy10, AAVrh32.33, clade F AAVHSC such as AAVHSC7, AAVHSC15 and AAVHSC17, tyrosine, lysine and serine capsid mutants of AAV serotypes; AAV hybrid serotypes, in particular AAV9.rh74 (WO 2019/193119); AAV hybrid serotypes comprising HVR sequences from AAV13 or hybrid AAV2/13 serotypes disclosed in WO 2022/003211A1; in particular HVR sequences from hybrid AAV2/13 capsids of SEQ ID NO: 2 to 30 disclosed in WO 2022/003211; more particularly AAV hybrid capsids of SEQ ID NO: 33 to 43, 45, 47 to 58 and 60 to 73 disclosed in WO 2022/003211.

In some particular embodiments, the peptide-modified AAV capsid protein is from AAV serotype selected from the group consisting of: AAV8, AAV9, AAVrh74, variants and hybrids thereof. AAV8 capsid corresponds to the amino acid sequence SEQ ID NO: 48. AAV9 capsid corresponds to the amino acid sequence GenBank accession number AY530579.1 accessed on 24 Jun. 2004 or SEQ ID NO: 49. AAVrh74 capsid corresponds to the amino acid sequence SEQ ID NO: 50. AAV8, AAV9 or AAVrh74 serotype includes the natural (wild-type) serotype as listed above (SEQ ID NO: 48 to 50) as well as any artificial serotype including any variant or hybrid derived from said serotype. The invention encompasses AAV8, AAV9 or AAVrh74 capsid or serotype having at least at least 85% identity with SEQ ID NO: 48 to 50; in particular having 90%, 95%, 96%, 97%, 98%, 99% or 100% identity with SEQ ID NO: 48 to 50. Particular hybrids are: (i) AAV9.rh74 disclosed in WO 2019/193119 and (ii) hybrids derived from AAV8, AAV9 or AAVrh74 serotypes comprising variable region(s) from AAV13 or hybrid AAV2/13 disclosed in WO 2022/003211A1; in particular HVR sequences from hybrid AAV2/13 capsids of SEQ ID NO: 2 to 30 disclosed in WO 2022/003211; more particularly AAV hybrid capsids of SEQ ID NO: 33 to 43, 45, 47 to 58 and 60 to 73 disclosed in WO 2022/003211. Preferred AAV hybrid serotype is AAV9.rh74 disclosed in WO 2019/193119; preferably comprising a sequence having at least 95% identity with SEQ ID NO: 51; more preferably comprising SEQ ID NO: 51.

The term “identity” refers to the sequence similarity between two polypeptide molecules or between two nucleic acid molecules. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wisconsin) pileup program, or any of sequence comparison algorithms such as BLAST, FASTA or CLUSTALW.

In some embodiments, the modified AAV capsid protein is from AAV serotype selected from the group consisting of: AAV8, AAV9, AAVrh74, variants and hybrids thereof as described herein; more preferably hybrid serotype AAV9.rh74 as described herein.

In some particular embodiments, the modified AAV capsid is from AAV9 or AAV9.rh74 and comprises the peptide P1, preferably inserted in the variable region VIII as described herein. In some more particular embodiments, the modified AAV capsid comprises a sequence having at least 95% identity with any one of SEQ ID NO: 52 to 56 which comprises said peptide P1; preferably comprising SEQ ID NO: 54.

### Tandem Promoter

The tandem promoter (or hybrid or tandem promoter) according to the present invention comprises a muscle-selective promoter fused to a liver-selective promoter. According to the present invention, transcription regulatory elements are selected for expression in muscles and in the liver. Expression is sought in the liver to induce immune tolerance to the transgene of interest while expression is sought in muscle to induce a therapeutic effect, in particular for treating muscle diseases.

In the context of the present invention, a “transcription regulatory element” is a DNA sequence able to drive or enhance transgene expression in a tissue or cell. In the context of the present invention, transcription regulatory elements are selected from tissue-selective promoters and tissue-selective enhancers. In a particular embodiment, the transcription regulatory elements are selected from tissue-selective promoters and tissue-selective enhancers of tissue-selective or tissue-specific genes.

According to the present invention “tissue-selectivity” means that a transcription regulatory element preferentially drives (in case of a promoter) or enhances (in case of an enhancer) expression of a gene operably linked to said transcription regulatory element in a given tissue, or set of tissues, as compared to expression in another tissue(s). This definition of “tissue-selectivity” does not exclude the possibility for a tissue-selective transcription regulatory element (such as a tissue-selective promoter) to leak to some extent. By “leak”, “leaking” or declinations thereof, it is meant the possibility for a transcription regulatory element selective of a one tissue to drive or increase expression of a transgene operably linked to said transcription regulatory element into another tissue, although at lower expression levels. For example, a muscle-selective promoter may leak in the liver tissue, meaning that expression drove from this promoter is higher in the muscle tissue than in the liver tissue. Alternatively, the tissue-selective transcription regulatory element may be a “tissue-specific” transcription regulatory element, meaning that this transcription regulatory element not only drives or enhances expression in a given tissue, or set of tissues, in a preferential manner, but also that this regulatory element does not, or does only marginally, drive or enhance expression in other tissues.

In the context of the present invention, the expression “tissue-selective promoters” includes natural or synthetic promoters. In particular, the expression “tissue-selective promoters” also denotes synthetic promoters comprising a tissue-selective promoter and an enhancer having the same tissue-selectivity as the promoter. An illustrative promoter encompassed by this expression is, for example, the fusion of the ApoE enhancer and the hAAT promoter, the fusion of which corresponding to a liver-selective promoter according to the definition provided in this paragraph.

Tissue-selective enhancers may be derived from cis-regulatory modules (CRMs) containing clusters of evolutionary conserved transcription factor binding site motifs (TFBS) associated with robust tissue-selective or tissue-specific expression.

In the context of the present invention, a “hybrid transcription regulatory element” denotes a DNA sequence able to drive a transgene expression in muscle and liver tissues in a tissue-dependent manner. According to the present invention, and as is explained in more details below, each transcription regulatory element is tissue- or cell-selective, i.e. it may drive expression of a transgene of interest in a tissue-selective manner, thereby preferentially restricting the expression of the transgene into tissues where the transgene product is desired (i.e., muscle and liver).

The tandem promoter may comprise a muscle-selective promoter as described herein, eventually combined with a muscle-selective enhancer as described herein.

One example of a suitable muscle-selective promoter includes a muscle creatine kinase (MCK) promoter. Non-limiting examples of suitable muscle creatine kinase promoters are human muscle creatine kinase promoters and truncated murine muscle creatine kinase [(tMCK) promoters] (Wang B et al, Construction and analysis of compact muscle-selective promoters for AAV vectors. Gene Ther. 2008 November; 15 (22): 1489-99) (representative GenBank Accession No. AF188002). Human muscle creatine kinase has the Gene ID No. 1158 (representative GenBank Accession No. NC_000019.9, accessed on Dec. 26, 2012). Other examples of muscle-selective promoters include a synthetic promoter C5.12 (spC5.12, alternatively referred to herein as “C5.12”), such as the spC5.12 shown in SEQ ID NO:1 or the spC5.12 promoter (disclosed in Wang et al., Gene Therapy volume 15, pages 1489-1499 (2008)), the MHCK7 promoter (Salva et al. Mol Ther. 2007 February; 15 (2): 320-9); myosin light chain (MLC) promoters, for example MLC2 (Gene ID No. 4633; representative GenBank Accession No. NG_007554.1, accessed on Dec. 26, 2012); myosin heavy chain (MHC) promoters, for example alpha-MHC (Gene ID No. 4624; representative GenBank Accession No. NG_023444.1, accessed on Dec. 26, 2012); desmin promoters (Gene ID No. 1674; representative GenBank Accession No. NG_008043.1, accessed on Dec. 26, 2012); cardiac troponin C promoters (Gene ID No. 7134; representative GenBank Accession No. NG_008963.1, accessed on Dec. 26, 2012); troponin I promoters (Gene ID Nos. 7135, 7136, and 7137; representative GenBank Accession Nos. NG_016649.1, NG_011621.1, and NG_007866.2, accessed on Dec. 26, 2012); myoD gene family promoters (Weintraub et al., Science, 251, 761 (1991); Gene ID No. 4654; representative GenBank Accession No. NM_002478, accessed on Dec. 26, 2012); alpha actin promoters (Gene ID Nos. 58, 59, and 70; representative GenBank Accession Nos. NG_006672.1, NG_011541.1, and NG_007553.1, accessed on Dec. 26, 2012); beta actin promoters (Gene ID No. 60; representative GenBank Accession No. NG_007992.1, accessed on Dec. 26, 2012); gamma actin promoters (Gene ID No. 71 and 72; representative GenBank Accession No. NG 011433.1 and NM_001199893, accessed on Dec. 26, 2012); muscle-selective promoters residing within intron 1 of the ocular form of Pitx3 (Gene ID No. 5309) (Coulon et al; the muscle-selective promoter corresponds to residues 11219-11527 of representative GenBank Accession No. NG_008147, accessed on Dec. 26, 2012); and the promoters described in US Patent Publication US 2003/0157064, and CK6 promoters (Wang et al 2008 doi: 10.1038/gt.2008.104). In particular embodiments, the muscle-selective promoter is the E-Syn promoter (sequence shown in SEQ ID NO:2) described in Wang et al., Gene Therapy volume 15, pages 1489-1499 (2008), comprising the combination of a MCK-derived enhancer and of the spC5.12 promoter. In particular embodiments of the invention, the muscle-selective promoter is selected from the group consisting of a spC5.12 promoter, the MHCK7 promoter, the E-syn promoter, a muscle creatine kinase myosin light chain (MLC) promoter, a myosin heavy chain (MHC) promoter, a desmin promoter, a cardiac troponin C promoter, a troponin I promoter, a myoD gene family promoter, an alpha actin promoter, a beta actin promoter, a gamma actin promoter, a muscle-selective promoter residing within intron 1 of the ocular form of Pitx3 and a CK6 promoter. In particular embodiments, the muscle-selective promoter is selected from the group consisting of the spC5.12, desmin and MCK promoters. In further embodiments, the muscle-selective promoter is selected from the group consisting of the spC5.12 and MCK promoters. In more particular embodiments, the muscle-selective promoter is the spC5.12 promoter. In specific embodiments, the muscle-selective promoter is not the desmin promoter.

Transcription regulatory elements that are, in particular, able to enhance muscle-selective expression of genes, in particular expression in cardiac muscle and/or skeletal muscle, are those disclosed in WO 2015/110449. Particular examples of nucleic acid transcription regulatory elements that comprise an artificial sequence include the transcription regulatory elements that are obtained by rearranging the transcription factor binding sites (TFBS) that are present in the sequences disclosed in WO 2015/110449. Said rearrangement may encompass changing the order of the TFBSs and/or changing the position of one or more TFBSs relative to the other TFBSs and/or changing the copy number of one or more of the TFBSs. For example, a nucleic acid transcription regulatory element for enhancing muscle-selective gene expression, in particular cardiac and skeletal muscle-selective gene expression, may comprise binding sites for E2A, HNH 1, NF1, C/EBP, LRF, MyoD, and SREBP; or for E2A, NF1, p53, C/EBP, LRF, and SREBP; or for E2A, HNH 1, HNF3a, HNF3b, NF1, C/EBP, LRF, MyoD, and SREBP; or E2A, HNF3a, NF1, C/EBP, LRF, MyoD, and SREBP; or for E2A, HNF3a, NF1, CEBP, LRF, MyoD, and SREBP; or for HNF4, NF1, RSRFC4, C/EBP, LRF, and MyoD, or NF1, PPAR, p53, C/EBP, LRF, and MyoD. In further examples, these nucleic acid transcription regulatory elements comprise at least two, such as 2, 3, 4, or more copies of one or more of the TFBSs recited before.

Other transcription regulatory elements that are, in particular, able to enhance muscle-selective expression of genes, when operably-linked to a muscle-selective promoter are the liver-selective enhancers disclosed in WO 2020/208032.

The tandem promoter may comprise a liver-selective promoter as described herein, and eventually a liver-selective enhancer as described herein. In particular embodiments, the hybrid-promoter comprises a combination of a liver-selective promoter and a liver-selective enhancer.

Illustrative liver-selective transcription regulatory elements include, without limitation, the Apolipoprotein E (ApoE—enhancer sequence shown in SEQ ID NO: 3) and A-I (Apo A-I) enhancers (Van Linthout S, Hum Gene Ther. 2002 May 1; 13 (7): 829-40), antitrypsin promoters—for example the alpha-1 antitrypsin promoter (hAAT—shown in SEQ ID NO: 4), transthyretin promoter (TTR), albumin promoter (Alb), thyroxine-binding globulin (TBG) promoter, the LSP promoter (comprising a thyroid hormone-binding globulin promoter sequence, two copies of an alpha1-microglobulin/bikunin enhancer sequence, and a leader sequence—Ill, Charles R., et al., 1997). Optimization of the human factor VIII complementary DNA expression plasmid for gene therapy of hemophilia A. Blood Coag. Fibrinol. 8: S23-S30.), etc. Other useful liver-selective promoters are known in the art, for example those listed in the Liver Specific Gene Promoter Database compiled by the Cold Spring Harbor Laboratory (http://rulai.cshl.edu/LSPD/). Composite or artificial liver promoters are derived by combining promoter regions of liver-expressed genes. Other transcription regulatory elements that are, in particular, able to enhance liver-selective expression of genes, are those disclosed in WO 2009/130208. In particular embodiments, the liver-selective transcription regulatory element comprises the combination of the ApoE enhancer with a liver-selective promoter selected from the group consisting of antitrypsin promoters—for example alpha-1 antitrypsin promoter (hAAT—shown in SEQ ID NO: 4), transthyretin promoter (TTR), albumin promoter (Alb), thyroxine-binding globulin (TBG) promoter, LSP promoter defined above, and any other liver-selective promoter such as those listed in the Liver Specific Gene Promoter Database compiled by the Cold Spring Harbor Laboratory (http://rulai.cshl.edu/LSPD/). In particular embodiments, the liver-selective transcription regulatory element for use in the context of the present invention is a liver-selective promoter selected from the group consisting of: an alpha-1 antitrypsin promoter (hAAT), a combination of the ApoE enhancer and an hAAT promoter, a transthyretin promoter, an albumin promoter, a thyroxine-binding globulin (TBG) promoter and a LSP promoter. A more particular liver-selective transcription regulatory element for use in the context of the invention is the combination of the ApoE enhancer (ApoE) and an hAAT promoter, in particular the combination of ApoE-enhancer sequence shown in SEQ ID NO: 3 and hAAT—shown in SEQ ID NO: 4.

The muscle-selective or liver-selective promoter may be a full-length promoter, a minimal promoter, or a modified promoter derived from any one of the genes disclosed herein. In particular embodiments, the promoter is a minimal-promoter derived from any one of the genes disclosed herein. In particular embodiments, the promoter is a human promoter derived from any one of the genes disclosed herein, preferably a human minimal-promoter derived from any one of the genes disclosed herein. In particular embodiments, the promoter is a modified promoter derived from any one of the genes disclosed herein.

In particular embodiments, the tandem promoter comprises a muscle-selective promoter fused to a combination of a liver-selective enhancer and a liver-selective promoter.

Selection of the transcription regulatory elements to be included in the tandem promoter of the invention will depend on the specific aim of the nucleic acid sequence and the transgene of interest operably linked to it. In particular, in case of the use of the nucleic acid sequence of the invention in a vector for gene therapy, it will depend on the disease or disorder the practitioner aims to treat. Depending on the case, the transcription regulatory elements may be selected as being capable of driving expression in a number of tissues or cells other than muscles and the liver, such as in the central nervous system such as in the brain, spinal cord, retina, cochlea, optic nerve, and/or olfactory nerves and epithelium for example in neurons (e.g. in motor neurons, sensory neurons or interneurons) or glial cells (oligodendrocytes, astrocytes, ependymal cells, microglia), in the peripheral nervous system (PNS), in the kidney, in the eye, or in the lung. Other tissues or cells of interest may include circulating cells such as cells of the immune system, for example in B cells, T cells or macrophages; hematopoietic cells; or endothelial cells. Transcription regulatory elements for expression into neurons are disclosed for example in WO 2019/154939.

CRMs useful in the practice of the present invention include those described in Rincon et al., Mol Ther. 2015 January; 23 (1): 43-52, Chuah et al., Mol Ther. 2014 September; 22 (9): 1605-13 or Nair et al., Blood. 2014 May 15; 123 (20): 3195-9.

The order of the muscle-selective, liver-selective, and eventually other tissue-selective transcription regulatory elements respectively one to another may vary. In particular embodiments, wherein the tandem promoter comprises a liver-specific promoter alone or in combination with a liver-specific enhancer, said transcription regulatory element(s) are located 5′ in relation to any other transcription regulatory element introduced in the tandem promoter of the invention.

In the context of the present invention, the transcription regulatory element introduced into the tandem promoter according to the invention may be either fused directly or linked via a linker. For example, in case of a design with two different promoters, a direct fusion means that the first nucleotide of the second promoter immediately follows the last nucleotide of the first promoter. In case of a link via a linker, a nucleotide sequence is present between the last nucleotide of the first promoter and the first nucleotide of the second promoter. For example, the length of the linker may be comprised between 1 and 50 nucleotides, such as from 1 to 40 nucleotides, such as from 1 to 30 nucleotides, such as from 1 to 20 nucleotides, such as from 1 to 10 nucleotides.

In a particular embodiment, the tandem promoter according to the invention comprises, in this order from 5′ to 3′:
the ApoE enhancer; and
a spC5.12 promoter.

In a variant of this embodiment, the tandem promoter according to the invention comprises a combination of SEQ ID NO: 3 and SEQ ID NO: 1, such as the sequence shown in SEQ ID NO:6.

In another particular embodiment, the tandem promoter according to the invention comprises in this order from 5′ to 3′:
the hAAT promoter; and
a spC5.12 promoter.

In a variant of this embodiment, the tandem promoter according to the invention comprises a combination of SEQ ID NO: 4 and SEQ ID NO: 1.

In another further particular embodiment, the tandem promoter according to the invention comprises in this order from 5′ to 3′:
the ApoE enhancer/hAAT promoter; and
a spC5.12 promoter.

In a particular variant of this embodiment, the tandem promoter according to the invention comprises a combination of SEQ ID NO: 3, SEQ ID NO: 4 and SEQ ID NO: 1, such as the sequence shown in SEQ ID NO: 6, named LiMP herein.

One example of preferred AAV vector according to the present invention comprises: (i) a tandem promoter comprising or consisting of SEQ ID NO: 6 and (ii) a peptide-modified AAV capsid comprising or consisting of SEQ ID NO: 54.

### Transgene Expression Cassette

The tandem promoter and transgene of interest according to the invention may be introduced into an expression cassette, designed for providing the expression of a transgene of interest into the target tissues of interest (i.e., muscles and liver and optionally other tissues such as nervous system as described herein).

The transgene expression cassette is a nucleic acid construct comprising the transgene operably linked to the tandem promoter. The term “operably linked” as used herein refers to the arrangement of various nucleic acid elements such that the elements are functionally connected and are able to interact with each other.

In some embodiments, the transgene is operably linked to further regulatory sequences capable of further controlling the expression of the transgene of interest by decreasing or suppressing its expression in certain tissues that are not of interest, of by stabilizing the mRNA encoded by the transgene of interest, i.e., coding for the protein or RNA of interest. These sequences include, without limitation, silencer (such as tissue-specific silencer), in particular microRNA target sequence; intron; transcription termination signal (polyadenylation signal), and post-transcriptional regulatory element, such as the Woodchuck hepatitis virus (WHV) post-transcriptional regulatory element (WPRE). Therefore, the transgene, tandem promoter and further regulatory sequences are included in a nucleic acid construct forming the transgene expression cassette.

In particular embodiments, the transgene of interest may be preceded by an intron, in particular an intron placed between the tandem promoter according to the invention and the transgene of interest. An intron may be introduced to increase mRNA stability and production of the protein of interest. In addition, a modified intron designed to decrease the number of, or even totally remove, alternative open reading frames (ARFs) found in said intron can significantly improve the expression of the transgene. Furthermore, by decreasing the number of ARFs within the intron included within the construct of the invention, it is believed that the construct immunogenicity is also decreased. Preferably, ARFs are removed whose length spans over 50 bp and have a stop codon in frame with a start codon. ARFs may be removed by way of nucleotide substitution, insertion or deletion, preferably by nucleotide substitution. For example, an ATG or a GTG may be replaced by a CTG, which is not a start codon, within the sequence of the intron of interest. Examples of introns which can be used in the present invention include those disclosed in WO 2020/212626, in particular human beta globin b2 (or HBB2) intron, modified HBB2 intron; a coagulation factor IX (FIX) intron, in particular hFIX, more particularly derived from first intron, and modified intron thereof; chicken beta-globin intron and modified intron thereof; and SV40 intron. In more particular embodiments, the AAV vector of the invention further comprises a SV40 intron inserted between the tandem promoter and the transgene of interest.

In particular embodiments, the transgene expression cassette further comprises a transcription termination signal (polyadenylation signal) operably linked to the transgene coding sequence (i.e., at the 3′-end of the coding sequence). Examples of polyA which can be used in the present invention include the bovine growth hormone (BGH, bgh or bGH) polyadenylation signal, the human beta globin b2 (HBB2) polyadenylation signal, and the Simian virus 40 (SV40) polyadenylation signal. In more particular embodiments, the AAV vector of the invention further comprises a bgh polyadenylation signal.

In particular embodiments, the AAV vector of the invention comprises in this order from 5′ to 3′: the tandem promoter as disclosed herein, in particular comprising: (i) a combination of SEQ ID NO: 3 and/or SEQ ID NO: 4, SEQ ID NO: 1; preferably SEQ ID NO: 5 or 6; (ii) an intron, such as an HBB2 or SV40 intron, in particular SV40 intron; (iii) the transgene of interest; and (iv) a polyadenylation signal, such as bgh polyadenylation signal.

According to the present invention, a “transgene of interest” refers to a polynucleotide sequence that encodes a RNA or protein product and that may be introduced into a cell for a sought purpose, and is capable of being expressed under appropriate conditions. A transgene of interest is a gene useful for a particular application, such as with no limitation, diagnosis, reporting, modifying, therapy and genome editing. For example, the gene of interest may be a therapeutic gene, a reporter gene or a genome-editing enzyme.

A therapeutic transgene is selected and used to lead to a desired therapeutic outcome, in particular for achieving expression of said therapeutic transgene into a cell, tissue or organ into which expression of said therapeutic transgene is needed (i.e., target cell, tissue or organ). Therapy may be achieved by a number of ways, including by expressing a protein into a cell that does not express said protein, by expressing a protein into a cell that expresses a mutated version of the protein, by expressing a protein that is toxic to the target cell into which it is expressed (strategy used, for example, for killing unwanted cells such as cancer cells), by expressing an antisense RNA to induce gene repression or exon skipping, by expressing a silencing RNA such as a shRNA whose purpose is to suppress the expression of a protein, or by expressing a genome-editing enzyme whose purpose is to modify a target genomic sequence.

The gene of interest is any nucleic acid sequence capable of modifying a target gene or target cellular pathway, in target cells, tissue or organ. For example, the gene may modify the expression, sequence or regulation of the target gene or cellular pathway. In some embodiments, the gene of interest is a functional version of a gene or a fragment thereof. The functional version of said gene includes the wild-type gene, a variant gene such as variants belonging to the same family and others, or a truncated version, which preserves the functionality of the encoded protein at least partially. A functional version of a gene is useful for replacement or additive gene therapy to replace a gene, which is deficient or non-functional in a patient. In other embodiments, the gene of interest is a gene which inactivates a dominant allele causing an autosomal dominant genetic disease. A fragment of a gene is useful as recombination template for use in combination with a genome editing enzyme.

The protein encoded by the transgene of interest is any protein or peptide of interest such as with no limitations a protein encoded by a functional version of a non-functional or deficient gene for replacement or additive gene therapy; an antibody or antibody fragment, a genome-editing enzyme, or another protein.

The RNA encoded by the transgene of interest is advantageously complementary to a target DNA or RNA sequence or binds to a target protein. For example, the RNA is an interfering RNA such as a shRNA, a microRNA, a guide RNA (gRNA) for use in combination with a Cas enzyme or similar enzyme for genome editing, an antisense RNA capable of exon skipping such as a modified small nuclear RNA (snRNA) or a long non-coding RNA. The interfering RNA or microRNA may be used to regulate the expression of a target gene having altered expression in a target cell, tissue or organ. The guide RNA in complex with a Cas enzyme or similar enzyme for genome editing may be used to modify the sequence of a target gene, in particular to correct the sequence of a mutated/deficient gene or to modify the expression of a target gene having altered expression in a target cell, tissue or organ. The antisense RNA capable of exon skipping is used in particular to correct a reading frame and restore expression of a deficient gene having a disrupted reading frame. In some embodiments, the RNA is a therapeutic RNA.

The genome-editing enzyme according to the invention is any enzyme or enzyme complex capable of modifying a target gene or target cellular pathway in target cells. For example, the genome-editing enzyme may modify the expression, sequence or regulation of the target gene or cellular pathway. The genome-editing enzyme is advantageously an engineered nuclease, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease such as Cas enzyme and similar enzymes, may be a functional nuclease which generates a double-strand break (DSB) or single-stranded DNA break (nickase such as Cas9 (D10A) in the target genomic locus and is used for site-specific genome editing applications, including with no limitations: gene correction, gene replacement, gene knock-in, gene knock-out, mutagenesis, chromosome translocation, chromosome deletion, and the like. For site-specific genome editing applications, the genome-editing enzyme, in particular an engineered nuclease such as Cas enzyme and similar enzymes may be used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene having altered expression in target cells, tissue or organ. The genome-editing enzyme, such as Cas enzyme and similar enzyme may be a DNA base-editor such as cytosine base-editor and adenine base-editor or a prime-editor. Base-editors can install all four transition mutations while Prime-editor expand the scope of donor-free precise DNA editing to not only all transition and transversion mutations, but small insertion and deletion mutations as well. Collectively, DNA base-editing and prime-editing tools enable precise nucleotide substitutions in a programmable manner, without requiring a donor template. Alternatively, the genome-editing enzyme, such as Cas enzyme and similar enzymes may be engineered to become nuclease-deficient and used as DNA-binding protein for various genome engineering applications in target cells, tissue or organ, such as with no limitation: transcriptional activation, transcriptional repression, epigenome modification, genome imaging, DNA or RNA pull-down and the like.

The transgene of interest is a functional gene able to produce the encoded protein, peptide or RNA in target cells, tissue or organ. In some embodiments, the gene of interest is a human gene. In some embodiments, the sequence of the gene of interest is optimized for expression in the treated individual, preferably a human individual. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites. Sequence optimization may also include reduction of sequence length. The transgene may comprise a shortened sequence to facilitate transgene cloning in rAAV vector or improve transgene expression in target cells or tissue.

The transgene of interest may encode a protein that remains in the target tissue after synthesis. Alternatively, the transgene may encode a protein that is secreted in the bloodstream after synthesis. To express proteins that are secreted in the bloodstream, the transgene advantageously comprises a signal peptide or signal sequence at the 5′end of the coding sequence. Signal peptides (SP) are short peptide sequences which are present at the N-terminus of secretory proteins and are used to target proteins for secretion. Multiple signal peptides are known in the art and publicly available (see in particular, Signal Peptide Website and SPdb sequence databases; Puzzo et al., Sci. Transl. Med., 2017, 9 (418): doi: 10.1126). Proteins that are secreted in the bloodstream may be expressed in the form of fusion proteins, wherein the protein of interest is linked to a protein stabilizing moiety or to a target cell receptor binding, to target the secreted protein from the bloodstream to a target organ.

The AAV vector (or AAV vector particle) according to the invention comprises the peptide-modified AAV capsid protein and the rAAV vector genome comprising the transgene expression cassette flanked by ITRs. The AAV vector particle according to the invention is suitable gene therapy, in particular gene therapy targeting muscles. The genome of the rAAV vector may either be a single-stranded or self-complementary double-stranded genome (McCarty et al, Gene Therapy, 2003 December, 10 (26), 2112-2118). Self-complementary vectors are generated by deleting the terminal resolution site (trs) from one of the AAV terminal repeats. These modified vectors, whose replicating genome is half the length of the wild-type AAV genome have the tendency to package DNA dimers. In particular embodiments, the AAV vector is a pseudotyped vector, i.e. its genome and capsid are derived from AAVs of different serotypes. In preferred embodiments, the genome of the pseudotyped vector is derived from AAV2. The rAAV vector particle may be obtained using standard AAV production methods that are well-known in the art (Review in Aponte-Ubillus et al., Applied Microbiology and Biotechnology, 2018, 102:1045-1054). AAV vectors are usually produced by co-transfecting cells suitable for AAV production with a plasmid containing recombinant AAV vector genome comprising the gene of interest inserted in an expression cassette, flanked by AAVITRs (AAV transfer plasmid), and plasmid(s) expressing AAV Rep and Cap proteins. Alternatively, producer cells which stably express AAV Rep and Cap proteins may be transfected with an AAV transfer plasmid. Briefly, following transfection with above plasmid(s) in the presence of sufficient helper function to permit packaging of the rAAV vector genome into AAV capsid particle, the cells are incubated for a time sufficient to allow the production of AAV vector particles, the cells are then harvested, lysed, and AAV vector particles are purified by standard purification methods such as affinity chromatography and Iodixanol or Cesium Chloride density gradient ultracentrifugation.

The invention also relates to an isolated cell, in particular a liver cell, a muscle cell or a combination thereof, which is which is genetically modified or transformed with an AAV vector of the invention.

### Pharmaceutical Compositions and Therapeutic Uses

Another aspect of the invention is a pharmaceutical composition comprising at least an active agent selected from an AAV vector particle or cell(s) of the invention, and a pharmaceutically acceptable carrier.

The AAV vector particle and derived cell(s) or pharmaceutical composition of the invention may be used for treating diseases by gene therapy, in particular targeted gene therapy directed to muscle cell, tissue or organ. The cell and derived pharmaceutical composition of the invention may be used for treating diseases by cell therapy, in particular cell therapy directed to muscle (i.e., muscle-directed cell therapy).

As used herein “Gene therapy” refers to a treatment of an individual which involves delivery of nucleic acid of interest into an individual's cells for the purpose of treating a disease. Delivery of the nucleic acid is generally achieved using a delivery vehicle, also known as a vector. The AAV vector particle of the invention may be employed to deliver a gene to a patient's cells.

As used herein “Cell therapy” refers to a process wherein cells stably transduced by a AAV vector of the invention are delivered to the individual in need thereof by any appropriate mean such as for example by intravenous injection (infusion), or injection in the tissue of interest (implantation or transplantation). In particular embodiments, cell therapy comprises collecting cells from the individual, modifying the individual's cells with the AAV vector of the invention, and administering the stably transduced cells back to the patient. As used herein “cell” refers to isolated cell, natural or artificial cellular aggregate, bioartificial cellular scaffold and bioartificial organ or tissue.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

The two main types of gene therapy are the following:
a therapy aiming to provide a functional replacement gene for a deficient/abnormal gene: this is replacement or additive gene therapy;
a therapy aiming at gene or genome editing: in such a case, the purpose is to provide to a cell the necessary tools to correct the sequence or modify the expression or regulation of a deficient/abnormal gene so that a functional gene is expressed or an abnormal gene is suppressed (inactivated): this is gene editing therapy.

In additive gene therapy, the gene of interest may be a functional version of a gene, which is deficient or mutated in a patient, as is the case for example in a genetic disease. In such a case, the gene of interest will restore the expression of a functional gene. Thus, by gene editing or gene replacement a correct version of this gene is provided in target cells, in particular hepatocytes of affected patients, this may contribute to effective therapies against the disease.

Gene or genome editing uses one or more gene(s) of interest, such as:
a gene encoding a therapeutic RNA as defined above such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); and
a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and maybe also a fragment of a functional version of a gene for use as recombination template, as defined above.

Gene therapy is used for treating various inherited (genetic) or acquired diseases or disorders affecting the structure or function of target tissue or organ. The diseases may be caused by trauma, infection, degeneration, structural or metabolic defects, tumors, inflammatory or autoimmune disorders, stroke or other causes.

In some particular embodiments, the disease affects muscles including skeletal or cardiac muscles; the disease may affect nervous system including brain or spinal cord, in addition to muscles including skeletal or cardiac muscles.

In some particular embodiments, the disease is a myopathy such as skeletal and/or cardiomyopathy, preferably a genetic myopathy.

In some particular embodiments, the disease is a neuromuscular disorder, preferably a genetic neuromuscular disorder. Neuromuscular disease or disorder (NMD) is a very broad term encompassing a range of conditions that impair the functioning of the muscles, either directly, being pathologies of the voluntary muscle, or indirectly, being pathologies of the peripheral nervous system or neuromuscular junctions. Neuromuscular diseases are a broadly defined group of disorders that all involve injury or dysfunction of peripheral nerves or muscle or neuromuscular junctions. The site of injury can be in the cell bodies (i.e., amyotrophic lateral sclerosis [ALS] or sensory ganglionopathies), axons (i.e., axonal peripheral neuropathies or brachial plexopathies), Schwann cells (i.e., chronic inflammatory demyelinating polyradiculoneuropathy), neuromuscular junction (i.e., myasthenia gravis or Lambert-Eaton myasthenic syndrome), muscle (i.e., inflammatory myopathy or muscular dystrophy), or any combination of these sites. Some neuromuscular diseases are also associated with central nervous system disease, such as ALS.

Examples of mutated genes in genetic myopathies including genetic neuromuscular diseases that can be targeted by gene therapy using the AAV vector of the invention are listed in the following tables:

### Muscular Dystrophies

| Gene | Protein |
|---|---|
| DMD | Dystrophin |
| EMD | Emerin |
| FHL1 | Four and a half LIM domain 1 |
| LMNA | Lamin A/C |
| SYNE1 | Spectrin repeat containing, nuclear envelope 1 (nesprin 1) |
| SYNE2 | Spectrin repeat containing, nuclear envelope 2 (nesprin 2) |
| TMEM43 | Transmembrane protein 43 |
| TOR1AIP1 | Torsin A interacting protein 1 |
| DUX4 | Double homeobox 4 |
| SMCHD1 | Structural maintenance of chromosomes flexible hinge |
| | domain containing 1 |
| PTRF | Polymerase I and transcript release factor |
| MYOT | Myotilin |
| CAV3 | Caveolin 3 |
| DNAJB6 | HSP-40 homologue, subfamily B, number 6 |
| DES | Desmin |
| TNPO3 | Transportin 3 |
| HNRNPDL | Heterogeneous nuclear ribonucleoprotein D-like |
| CAPN3 | Calpain 3 |
| DYSF | Dysferlin |
| SGCG | Gamma sarcoglycan |
| SGCA | Alpha sarcoglycan |
| SGCB | Beta sarcoglycan |
| SGCD | Delta-sarcoglycan |
| TCAP | Telethonin |
| TRIM32 | Tripartite motif-containing 32 |
| FKRP | Fukutin-related protein |
| TTN | Titin |
| POMT1 | Protein-O-mannosyltransferase 1 |
| ANO5 | Anoctamin 5 |
| FKTN | Fukutin |
| POMT2 | Protein-O-mannosyltransferase 2 |
| POMGNT1 | O-linked mannose beta1,2-N-acetylglucosaminyltransferase |
| PLEC | Plectin |
| TRAPPC11 | trafficking protein particle complex 11 |
| GMPPB | GDP-mannose pyrophosphorylase B |
| DAG1 | Dystroglycan1 |
| DPM3 | Dolichyl-phosphate mannosyltransferase polypeptide 3 |
| ISPD | Isoprenoid synthase domain containing |
| VCP | Valosin-containing protein |
| LIMS2 | LIM and senescent cell antigen-like domains 2 |
| GAA | Glucosidase alpha, acid |

### Congenital Muscular Dystrophies

| Gene | Protein |
|---|---|
| LAMA2 | Laminin alpha 2 chain of merosin |
| COL6A1 | Alpha 1 type VI collagen |
| COL6A2 | Alpha 2 type VI collagen |
| COL6A3 | Alpha 3 type VI collagen |
| SEPN1 | Selenoprotein N1 |
| FHL1 | Four and a half LIM domain 1 |
| ITGA7 | Integrin alpha 7 precursor |
| DNM2 | Dynamin 2 |
| TCAP | Telethonin |
| LMNA | Lamin A/C |
| FKTN | Fukutin |
| POMT1 | Protein-O-mannosyltransferase 1 |
| POMT2 | Protein-O-mannosyltransferase 2 |
| FKRP | Fukutin-related protein |
| POMGNT1 | O-linked mannose beta1,2-N- |
| | acetylglucosaminyltransferase |
| ISPD | Isoprenoid synthase domain containing |
| POMGNT2 | protein O-linked mannose N- |
| | acetylglucosaminyltransferase 2 |
| B3GNT1 | UDP-GlcNAc:betaGal beta-1,3-N- |
| | acetylglucosaminyl-transferase 1 |
| GMPPB | GDP-mannose pyrophosphorylase B |
| LARGE | Like-glycosyltransferase |
| DPM1 | Dolichyl-phosphate mannosyltransferase 1, |
| | catalytic subunit |
| DPM2 | Dolichyl-phosphate mannosyltransferase |
| | subunitpolypeptide 2, regulatory |
| ALG13 | UDP-N-acetylglucosami-nyltransferase |
| B3GALNT2 | Beta-1,3-N-acetylgalacto-saminyltransferase 2 |
| TMEM5 | Transmembrane protein 5 |
| POMK | Protein-O-mannose kinase |
| CHKB | Choline kinase beta |
| ACTA1 | Alpha actin, skeletal muscle |
| TRAPPC11 | trafficking protein particle complex 11 |

### Congenital Myopathies

| Gene | Protein |
|---|---|
| TPM3 | Tropomyosin 3 |
| NEB | Nebulin |
| ACTA1 | Alpha actin, skeletal muscle |
| TPM2 | Tropomyosin 2 (beta) |
| TNNT1 | Slow troponin T |
| KBTBD13 | Kelch repeat and BTB (POZ) domain containing 13 |
| CFL2 | Cofilin 2 (muscle) |
| KLHL40 | Kelch-like family member 40 |
| KLHL41 | Kelch-like family member 41 |
| LMOD3 | Leiomodin 3 (fetal) |
| SEPN1 | Selenoprotein N1 |
| RYR1 | Ryanodine receptor 1 (skeletal) |
| MYH7 | Myosin, heavy polypeptide 7, cardiac muscle, beta |
| MTM1 | Myotubularin |
| DNM2 | Dynamin 2 |
| BIN1 | Amphiphysin |
| TTN | Titin |
| SPEG | SPEG complex locus |
| MEGF10 | Multiple EGF-like-domains 10 |
| MYH2 | Myosin, heavy polypeptide 2, skeletal muscle |
| MYBPC3 | Cardiac myosin binding protein-C |
| CNTN1 | Contactin-1 |
| TRIM32 | Tripartite motif-containing 32 |
| PTPLA | Protein tyrosine phosphatase-like |
| | (3-Hydroxyacyl-CoA dehydratase |
| CACNA1S | Calcium channel, voltage-dependent, L type, |
| | alpha 1S subunit |

### Distal Myopathies

| Gene | |
|---|---|
| symbol | protein |
| DYSF | Dysferlin |
| TTN | Titin |
| GNE | UDP-N-acetylglucosamine-2- epimerase/N- |
| | acetylmannosamine kinase |
| MYH7 | Myosin, heavy polypeptide 7, cardiac muscle, beta |
| MATR3 | Matrin 3 |
| TIA1 | Cytotoxic granuleassociated RNA binding protein |
| MYOT | Myotilin |
| NEB | Nebulin |
| CAV3 | Caveolin 3 |
| LDB3 | LIM domain binding 3 |
| ANO5 | Anoctamin 5 |
| DNM2 | Dynamin 2 |
| KLHL9 | Kelch-like homologue 9 |
| FLNC | Filamin C, gamma (actin-binding protein - 280) |
| VCP | Valosin-containing protein |

### Other Myopathies

| Gene | |
|---|---|
| symbol | protein |
| ISCU | Iron-sulfur cluster scaffold homolog (E. coli) |
| MSTN | Myostatin |
| FHL1 | Four and a half LIM domain 1 |
| BAG3 | BCL2-associated athanogene 3 |
| ACVR1 | Activin A receptor, type II-like kinase 2 |
| MYOT | Myotilin |
| FLNC | Filamin C, gamma (actin-binding protein - 280) |
| LDB3 | LIM domain binding 3 |
| LAMP2 | Lysosomal-associated membrane protein 2 precursor |
| VCP | Valosin-containing protein |
| CAV3 | Caveolin 3 |
| SEPN1 | Selenoprotein N1 |
| CRYAB | Crystallin, alpha B |
| DES | Desmin |
| VMA21 | VMA21 Vacuolar H+-ATPase Homolog (S. Cerevisiae) |
| PLEC | plectin |
| PABPN1 | Poly(A) binding protein, nuclear 1 |
| TTN | Titin |
| RYR1 | Ryanodine receptor 1 (skeletal) |
| CLN3 | Ceroid-lipofuscinosis, neuronal 3 (=battenin) |
| TRIM54 | |
| TRIM63 | Tripartite motif containing 63, E3 ubiquitin protein ligase |

### Myotonic Syndromes

| Gene | protein |
|---|---|
| DMPK | Myotonic dystrophy protein kinase |
| CNBP (ZNF9) | Cellular nucleic acid-binding protein |
| CLCN1 | Chloride channel 1, skeletal muscle (Thomsen |
| | disease, autosomal dominant) |
| CAV3 | Caveolin 3 |
| HSPG2 | Perlecan |
| ATP2A1 | ATPase, Ca++ transporting, fast twitch 1 |

### Ion Channel Muscle Diseases

| Gene | protein |
|---|---|
| CLCN1 | Chloride channel 1, skeletal muscle (Thomsen |
| | disease, autosomal dominant) |
| SCN4A | Sodium channel, voltage-gated, type IV, alpha |
| SCN5A | Voltage-gated sodium channel type V alpha |
| CACNA1S | Calcium channel, voltage-dependent, L type, |
| | alpha 1S subunit |
| CACNA1A | Calcium channel, voltage-dependent, P/Q type, |
| | alpha 1A subunit |
| KCNE3 | Potassium voltage-gated channel, Isk-related |
| | family, member 3 |
| KCNA1 | Potassium voltage-gated channel, shaker-related |
| | subfamily, member 1 |
| KCNJ18 | Kir2.6 (inwardly rectifying potassium channel 2.6) |
| KCNJ2 | Potassium inwardly-rectifying channel J2 |
| KCNH2 | Voltage-gated potassium channel, subfamily H, |
| | member 2 |
| KCNQ1 | Potassium voltage-gated channel, KQT-like |
| | subfamily, member 1 |
| KCNE2 | Potassium voltage-gated channel, Isk-related |
| | family, member 2 |
| KCNE1 | Potassium voltage-gated channel, Isk-related |
| | family, member 1 |

### Malignant Hyperthermia

| Gene | protein |
|---|---|
| RYR1 | Ryanodine receptor 1 (skeletal) |
| CACNA1S | Calcium channel, voltage-dependent, |
| | L type, alpha 1S subunit |

### Metabolic Myopathies

| Gene | protein |
|---|---|
| GAA | Acid alpha-glucosidase preproprotein |
| AGL | Amylo-1,6-glucosidase, 4-alpha-glucanotransferase |
| GBE1 | Glucan (1,4-alpha-), branching enzyme 1 (glycogen |
| | branching enzyme, Andersen disease, glycogen |
| | storage disease type IV) |
| PYGM | Glycogen phosphorylase |
| PFKM | Phosphofructokinase, muscle |
| PHKA1 | Phosphorylase b kinase, alpha submit |
| PGM1 | Phosphoglucomutase 1 |
| GYG1 | Glycogenin 1 |
| GYS1 | Glycogen synthase 3 glycogen synthase 1 (muscle) |
| | glycogen synthase 1 (muscle) |
| PRKAG2 | Protein kinase, AMP-activated, gamma 2 |
| | non-catalytic subunit |
| RBCK1 | RanBP-type and C3HC4-type zinc finger containing |
| | 1 (heme-oxidized IRP2 ubiquitin ligase 1) |
| PGK1 | Phosphoglycerate kinase 1 |
| PGAM2 | Phosphoglycerate mutase 2 (muscle) |
| LDHA | Lactate dehydrogenase A |
| ENO3 | Enolase 3, beta muscle specific |
| CPT2 | Carnitine palmitoyltransferase II |
| SLC22A5 | Solute carrier family 22 member 5 |
| SLC25A20 | Carnitine-acylcarnitine translocase |
| ETFA | Electron-transfer-flavoprotein, alpha polypeptide |
| ETFB | Electron-transfer-flavoprotein, beta polypeptide |
| ETFDH | Electron-transferring-flavoprotein dehydrogenase |
| ACADVL | Acyl-Coenzyme A dehydrogenase, very long chain |
| ABHD5 | Abhydrolase domain containing 5 |
| PNPLA2 | Adipose triglyceride lipase (desnutrin) |
| LPIN1 | Lipin 1 (phosphatidic acid phosphatase 1) |
| PNPLA8 | Patatin-like phospholipase domain containing 8 |

### Hereditary Cardiomyopathies

| Gene | protein |
|---|---|
| MYH6 | Myosin heavy chain 6 |
| MYH7 | Myosin, heavy polypeptide 7, cardiac muscle, beta |
| TNNT2 | Troponin T2, cardiac |
| TPM1 | Tropomyosin 1 (alpha) |
| MYBPC3 | Cardiac myosin binding protein-C |
| PRKAG2 | Protein kinase, AMP-activated, gamma 2 |
| | non-catalytic subunit |
| TNNI3 | Troponin I, cardiac |
| MYL3 | Myosin light chain 3 |
| TTN | Titin |
| MYL2 | Myosin light chain 2 |
| ACTC1 | Actin, alpha, cardiac muscle precursor |
| CSRP3 | Cysteine and glycine-rich protein 3 |
| | (cardiac LIM protein) |
| TNNC1 | Slow troponin C |
| VCL | Vinculin |
| MYLK2 | Myosin light chain kinase 2 |
| CAV3 | Caveolin 3 |
| MYOZ2 | Myozenin 2, or calsarcin 1, a Z disk protein |
| JPH2 | Junctophilin-2 |
| PLN | Phospholamban |
| NEXN | Nexilin(F-actin binding protein) |
| ANKRD1 | Ankyrin repeat domain 1 (cardiac muscle) |
| ACTN2 | Actinin alpha2 |
| NDUFAF1 | NADH-ubiquinone oxidoreductase 1 alpha |
| | subcomplex |
| TSFM | Ts translation elongation factor, mitochondrial |
| AARS2 | Alanyl-tRNA synthetase 2, mitochondrial |
| MRPL3 | Mitochondrial ribosomal protein L3 |
| COX15 | COX15 homolog, cytochrome c oxidase assembly |
| | protein (yeast) |
| MTO1 | Mitochondrial tRNA translation optimization 1 |
| MRPL44 | Mitochondrial ribosomal protein L44 |
| LMNA | Lamin A/C |
| LDB3 | LIM domain binding 3 |
| SCN5A | Voltage-gated sodium channel type V alpha |
| DES | Desmin |
| EYA4 | Eyes absent 4 |
| SGCD | Delta-sarcoglycan |
| TCAP | Telethonin |
| ABCC9 | ATP-binding cassette, sub-family C (member 9) |
| TMPO | Lamina-associated polypeptide 2 |
| PSEN2 | Presenilin 2 |
| CRYAB | Crystallin, alpha B |
| FKTN | Fukutin |
| TAZ | Tafazzin |
| DMD | Dystrophin |
| LAMA4 | Laminin alpha 4 |
| ILK | Integrin-linked kinase |
| MYPN | Myopalladin |
| RBM20 | RNA binding motif protein 20 |
| SYNE1 | Spectrin repeat containing, nuclear envelope 1 |
| | (nesprin 1) |
| MURC | Muscle-related coiled-coil protein |
| DOLK | Dolichol kinase |
| GATAD1 | GATA zinc finger domain containing 1 |
| SDHA | succinate dehydrogenase complex, subunit A, |
| | flavoprotein (Fp) |
| GAA | Acid alpha-glucosidase preproprotein |
| DTNA | Dystrobrevin, alpha |
| FLNA | Filamin A, alpha (actin binding protein 280) |
| TGFB3 | Transforming growth factor, beta 3 |
| RYR2 | Ryanodine receptor 2 |
| TMEM43 | Transmembrane protein 43 |
| DSP | Desmoplakin |
| PKP2 | Plakophilin 2 |
| DSG2 | Desmoglein 2 |
| DSC2 | Desmocollin 2 |
| JUP | Junction plakoglobin |
| CASQ2 | Calsequestrin 2 (cardiac muscle) |
| KCNQ1 | Potassium voltage-gated channel, KQT-like |
| | subfamily, member 1 |
| KCNH2 | Voltage-gated potassium channel, subfamily H, |
| | member 2 |
| ANK2 | Ankyrin 2 |
| KCNE1 | Potassium voltage-gated channel, Isk-related family, |
| | member 1 |
| KCNE2 | Potassium voltage-gated channel, Isk-related family, |
| | member 2 |
| KCNJ2 | Potassium inwardly-rectifying channel J2 |
| CACNA1C | Calcium channel, voltage-dependent, L type, alpha |
| | 1C subunit |
| SCN4B | Sodium channel, voltage-gated, type IV, beta subunit |
| AKAP9 | A kinase (PRKA) anchor protein (yotiao) 9 |
| SNTA1 | Syntrophin, alpha 1 |
| KCNJ5 | Potassium inwardly-rectifying channel, subfamily J, |
| | member 5 |
| NPPA | Natriuretic peptide precursor A |
| KCNA5 | Potassium voltage-gated channel, shaker-related |
| | subfamily, member 5 |
| GJA5 | Connexin 40 |
| SCN1B | Sodium channel, voltage-gated, type I, beta subunit |
| SCN2B | Sodium channel, voltage-gated, type II, beta subunit |
| NUP155 | Nucleoporin 155 kDa |
| GPD1L | Glycerol-3-phosphate dehydrogenase 1-like |
| CACNB2 | Calcium channel, voltage-dependent, beta 2 subunit |
| KCNE3 | Potassium voltage-gated channel, Isk-related family, |
| | member 3 |
| SCN3B | Sodium channel, voltage-gated, type III, beta subunit |
| HCN4 | Hyperpolarization activated cyclic nucleotide-gated |
| | potassium channel 4 |

### Congenital Myasthenic Syndromes

| Gene | protein |
|---|---|
| CHRNA1 | Cholinergic receptor, nicotinic, alpha polypeptide 1 |
| CHRNB1 | Cholinergic receptor, nicotinic, beta 1 muscle |
| CHRND | Cholinergic receptor, nicotinic, delta |
| CHRNE | Cholinergic receptor, nicotinic, epsilon |
| RAPSN | Rapsyn |
| CHAT | Choline acetyltransferase isoform |
| COLQ | Acetylcholinesterase collagen-like tail subunit |
| MUSK | muscle, skeletal, receptor tyrosine kinase |
| DOK7 | Docking protein 7 |
| AGRN | Agrin |
| GFPT1 | Glutamine-fructose-6-phosphate transaminase 1 |
| DPAGT1 | Dolichyl-phosphate (UDP-N-acetylglucosamine) N- |
| | acetylglucosaminephosphotransferase 1 |
| | (GlcNAc-1-P transferase) |
| LAMB2 | Laminin, beta 2 (laminin S) |
| SCN4A | Sodium channel, voltage-gated, type IV, alpha |
| CHRNG | Cholinergic receptor, nicotinic, gamma polypeptide |
| PLEC | plectin |
| ALG2 | Alpha-1,3/1,6-mannosyltransferase |
| ALG14 | UDP-N-acetylglucosaminyltransferase |
| SYT2 | Synaptotagmin II |
| PREPL | Prolyl endopeptidase-like |

### Spinal Muscular Atrophies (SMAs) & Motor Neuron Diseases

| Gene | protein |
|---|---|
| SMN1 | Survival of motor neuron 1, telomeric |
| IGHMBP2 | Immunoglobulin mu binding protein 2 |
| PLEKHG5 | Pleckstrin homology domain containing, family |
| | G (with RhoGef domain) member 5 |
| HSPB8 | Heat shock 27 kDa protein 8 |
| HSPB1 | Heat shock 27 kDa protein 1 |
| HSPB3 | Heat shock 27 kDa protein 3 |
| AARS | Alanyl-tRNA synthetase |
| GARS | Glycyl-tRNA synthetase |
| BSCL2 | Seipin |
| REEP1 | Receptor accessory protein 1 |
| SLC5A7 | Solute carrier family 5 (sodium/choline |
| | cotransporter), member 7 |
| DCTN1 | Dynactin 1 |
| UBA1 | Ubiquitin-activating enzyme 1 |
| ATP7A | ATPase, Cu++ transporting, alpha polypeptide |
| DNAJB2 | DnaJ (Hsp40) homolog, subfamily B, member 2 |
| TRPV4 | Transient receptor potential cation channel, |
| | subfamily V, member 4 |
| DYNC1H1 | Dynein, cytoplasmic 1, heavy chain 1 |
| BICD2 | Bicaudal D homolog 2 (Drosophila) |
| FBXO38 | F-box protein 38 |
| ASAH1 | N-acylsphingosine amidohydrolase (acid |
| | ceramidase) 1 |
| VAPB | Vesicle-associated membrane protein-associated |
| | protein B and C |
| EXOSC8 | Exosome component 8 |
| SOD1 | Superoxide dismutase 1, soluble |
| ALS2 | Alsin |
| SETX | Senataxin |
| FUS | Fusion (involved in t(12; 16) in malignant |
| | liposarcoma) |
| ANG | Angiogenin |
| TARDBP | TAR DNA binding protein |
| FIG4 | Sac domain-containing inositol phosphatase 3 |
| OPTN | Optineurin |
| ATXN2 | Ataxin 2 |
| VCP | Valosin-containing protein |
| UBQLN2 | Ubiquilin 2 |
| SIGMAR1 | Sigma non-opioid intracellular receptor 1 |
| CHMP2B | Charged multivesicular body protein 2B |
| PFN1 | Profilin 1 |
| MATR3 | Matrin 3 |
| NEFH | Neurofilament, heavy polypeptide |
| PRPH | Peripherin |
| C9orf72 | Chromosome 9 open reading frame 72 |
| CHCHD10 | Coiled-coil-helix-coiled-coil-helix domain |
| | containing 10 |
| SQSTM1 | Sequestosome 1 |
| AR | Androgen receptor |
| GLE1 | GLE1 RNA export mediator homolog (yeast) |
| ERBB3 | V-erb-b2 erythroblastic leukemia viral oncogene |
| | homolog 3 (avian) |
| PIP5K1C | Phosphatidylinositol-4-phosphate 5-kinase, |
| | type I, gamma |
| EXOSC3 | Exosome component 3 |
| VRK1 | Vaccinia related kinase 1 |
| SLC52A3 | Solute carrier family 52, riboflavin transporter, |
| | member 3 |
| SLC52A2 | Solute carrier family 52, riboflavin transporter, |
| | member 2 |
| HEXB | Hexosaminidase B |

### Hereditary Motor and Sensory Neuropathies

| Gene | Protein |
|---|---|
| PMP22 | Peripheral myelin protein 22 |
| MPZ | Myelin protein zero |
| LITAF | Lipopolysaccharide-induced TNF factor |
| EGR2 | Early growth response 2 protein |
| NEFL | Neurofilament, light polypeptide 68 kDa |
| HOXD10 | Homeobox D10 |
| ARHGEF10 | Rho guanine nucleotide exchange factor 10 |
| FBLN5 | Fibulin 5 (extra-cellular matrix) |
| DNM2 | Dynamin 2 |
| YARS | Tyrosyl-tRNA synthetase |
| INF2 | Inverted formin 2 |
| GNB4 | Guanine nucleotidebinding protein, beta-4 |
| GDAP1 | Ganglioside-induced differentiation-associated protein 1 |
| MTMR2 | Myotubularin-related protein 2 |
| SBF2 | SET binding factor 2 |
| SBF1 | SET binding factor 1 |
| SH3TC2 | KIAA1985 protein |
| NDRG1 | N-myc downstream regulated gene 1 |
| PRX | Periaxin |
| HK1 | Hexokinase 1 |
| FGD4 | Actin-filament binding protein Frabin |
| FIG4 | Sac domain-containing inositol phosphatase 3 |
| SURF1 | surfeit 1 |
| GJB1 | Gap junction protein, beta 1, 32 kDa (connexin 32) |
| AIFM1 | Apoptosis-inducing factor, mitochondrionassociated 1 |
| PRPS1 | Phosphoribosyl pyrophosphate synthetase 1 |
| PDK3 | Pyruvate dehydrogenase kinase, isoenzyme 3 |
| KIF1B | Kinesin family member 1B |
| MFN2 | Mitofusin 2 |
| RAB7A | RAB7, member RAS oncogene family |
| TRPV4 | Transient receptor potential cation channel, subfamily |
| | V, member 4 |
| GARS | Glycyl-tRNA synthetase |
| HSPB1 | Heat shock 27 kDa protein 1 |
| HSPB8 | Heat shock 27 kDa protein 8 |
| AARS | Alanyl-tRNA synthetase |
| DYNC1H1 | Dynein, cytoplasmic 1, heavy chain 1 |
| LRSAM1 | leucine rich repeat and sterile alpha motif containing 1 |
| DHTKD1 | dehydrogenase E1 and transketolase domain containing 1 |
| TRIM2 | Tripartite motif containing 2 |
| TFG | TRK-fused gene |
| MARS | methionyl-tRNA synthetase |
| KIF5A | Kinesin family member 5A |
| LMNA | Lamin A/C |
| MED25 | Mediator complex subunit 25 |
| DNAJB2 | DnaJ (Hsp40) homolog, subfamily B, member 2 |
| HINT1 | Histidine triad nucleotide binding protein 1 |
| KARS | Lysyl-tRNA synthetase |
| PLEKHG5 | Pleckstrin homology domain containing, family G |
| | (with RhoGef domain) member 5 |
| COX6A1 | Cytochrome c oxidase subunit VIa polypeptide 1 |
| IGHMBP2 | Immunoglobulin mu binding protein 2 |
| SPTLC1 | Serine palmitoyltransferase subunit 1 |
| SPTLC2 | Serine palmitoyltransferase long chain base subunit 2 |
| ATL1 | Atlastin GTPase 1 |
| KIF1A | Kinesin family member 1A |
| WNK1 | WNK lysine deficient protein kinase 1 |
| IKBKAP | Inhibitor of kappa light polypeptide gene enhancer in |
| | B-cells, kinase complex-associated protein |
| NGF | Nerve growth factor (beta polypeptide) |
| DNMT1 | DNA (cytosine-5)-methyltransferase 1 |
| SLC12A6 | Potassium chloride cotransporter KCC3 |
| GJB3 | Gap junction protein, beta 3, 31 kDa (=connexin 31) |
| sept-09 | Septin 9 |
| GAN | Gigaxonin |
| CTDP1 | CTD phosphatase subunit 1 |
| VRK1 | Vaccinia related kinase 1 |

### Hereditary Paraplegia

| Gene | |
|---|---|
| symbol | protein |
| ATL1 | Atlastin |
| SPAST | Spastin |
| NIPA1 | Non-imprinted in Prader-Willi/Angelman syndrome 1 |
| KIAA0196 | Strumpellin |
| KIF5A | Kinesin family member 5A |
| RTN2 | Reticulon 2 |
| HSPD1 | Heat shock 60 kDa protein 1 (chaperonin) |
| BSCL2 | Seipin |
| REEP1 | Receptor accessory protein 1 |
| ZFYVE27 | Protrudin |
| SLC33A1 | Solute carrier family 33 (acetyl- CoA transporter) |
| CYP7B1 | Cytochrome P450, family 7, subfamily B, polypeptide 1 |
| SPG7 | Paraplegin |
| SPG11 | Spatacsin |
| ZFYVE26 | Spastizin |
| ERLIN2 | ER lipid raft associated 2 |
| SPG20 | Spartin |
| SPG21 | Maspardin |
| B4GALNT1 | beta-1,4-N-acetyl-galactosaminyl transferase 1 |
| DDHD1 | DDHD domain containing 1 |
| KIF1A | Kinesin family member 1A |
| FA2H | Fatty acid 2-hydroxylase |
| PNPLA6 | Patatin-like phospholipase domain containing 6 |
| C19orf12 | chromosome 19 open reading frame 12 |
| GJC2 | gap junction protein, gamma 2, 47 kDa |
| NT5C2 | 5′-nucleotidase, cytosolic II |
| GBA2 | glucosidase, beta (bile acid) 2 |
| AP4B1 | adaptor-related protein complex 4, beta 1 subunit |
| AP5Z1 | Hypothetical protein LOC9907 |
| TECPR2 | tectonin beta-propeller repeat containing 2 |
| AP4M1 | Adaptor-related protein complex 4, mu 1 subunit |
| AP4E1 | Adaptor-related protein complex 5, zeta 1 subunit |
| AP4S1 | adaptor-related protein complex 4, sigma 1 subunit |
| DDHD2 | DDHD domain containing 2 |
| C12orf65 | adaptor-related protein complex 4, sigma 1 subunit |
| CYP2U1 | cytochrome P450, family 2, subfamily U, polypeptide 1 |
| ARL6IP1 | ADP-ribosylation factor-like 6 interacting protein 1 |
| AMPD2 | adenosine monophosphate deaminase 2 |
| ENTPD1 | ectonucleoside triphosphate diphosphohydrolase 1 |
| ALDH3A2 | Aldehyde dehydrogenase 3A2 |
| ALS2 | Alsin |
| L1CAM | L1 cell adhesion molecule |
| PLP1 | Proteolipid protein 1 |
| MTPAP | mitochondrial poly(A) polymerase |
| AFG3L2 | AFG3 ATPase family gene 3-like 2 (S. cerevisiae) 1 |
| SACS | Sacsin |

### Other Neuromuscular Disorders

| Gene | protein |
|---|---|
| TOR1A | Torsin A |
| SGCE | Sarcoglycan, epsilon |
| IKBKAP | Inhibitor of kappa light polypeptide gene enhancer in |
| | B-cells, kinase complex-associated protein |
| TTR | Transthyretin (prealbumin, amyloidosis type I) |
| KIF21A | Kinesin family member 21A |
| PHOX2A | Paired-like aristaless homeobox protein 2A |
| TUBB3 | Tubulin, beta 3 |
| TPM2 | Tropomyosin 2 (beta) |
| MYH3 | Myosine, heavy chain 3, skeletal muscle, embryonic |
| TNNI2 | Troponin I, type 2 |
| TNNT3 | Troponin T3, skeletal |
| SYNE1 | Spectrin repeat containing, nuclear envelope 1 |
| | (nesprin 1) |
| MYH8 | Myosin heavy chain, 8, skeletal muscle, perinatal |
| POLG | Polymerase (DNA directed), gamma |
| SLC25A4 | Mitochondrial carrier; adenine nucleotide translocator |
| C10orf2 | chromosome 10 open reading frame 2 |
| POLG2 | Mitochondrial DNA polymerase, accessory subunit |
| RRM2B | Ribonucleotide reductase M2 B (TP53 inducible) |
| TK2 | Thymidine kinase 2, mitochondrial |
| SUCLA2 | Succinate-CoA ligase, ADP-forming, beta subunit |
| OPA1 | optic atrophy 1 |
| STIM1 | Stromal interaction molecule 1 |
| ORAI1 | ORAI calcium release-activated calcium modulator 1 |
| PUS1 | Pseudouridylate synthase 1 |
| CHCHD10 | Coiled-coil-helix-coiled-coil-helix domain |
| | containing 10 |
| CASQ1 | Calsequestrin 1 (fast-twitch, skeletal muscle) |
| YARS2 | tyrosyl-tRNA synthetase 2, mitochondrial |

### Hereditary Ataxia

| Gene | |
|---|---|
| symbol | protein |
| ATXN1 | Ataxin 1 |
| ATXN2 | Ataxin 2 |
| ATXN3 | Ataxin 3 |
| SPTBN2 | Spectrin, beta, non-erythrocytic 2 |
| CACNA1A | Calcium channel, voltage-dependent, P/Q type, alpha 1A |
| | subunit |
| ATXN7 | Ataxin 7 |
| ATXN8OS | Ataxin 8 opposite strand |
| ATXN10 | Ataxin 10 |
| TTBK2 | Tau tubulin kinase 2 |
| PPP2R2B | Protein phosphatase 2 regulatory subunit B, beta isoform |
| KCNC3 | Potassium voltage-gated channel, Shaw-related subfamily, |
| | member 3 |
| PRKCG | Protein kinase C, gamma |
| ITPR1 | Inositol 1,4,5-triphosphate receptor type 1 |
| TBP | TATA box binding protein |
| IFRD1 | Interferon-related developmental regulator 1 |
| KCND3 | Potassium voltage-gated channel, Shal-related subfamily, |
| | member 3 |
| PDYN | prodynorphin |
| EEF2 | Eukaryotic translation elongation factor 2 |
| FGF14 | Fibroblast growth factor 14 |
| AFG3L2 | AFG3 ATPase family gene 3-like 2 (S. cerevisiae) 1 |
| BEAN1 | Brain expressed, associated with Nedd42 |
| TK2 | Thymidine kinase 2, mitochondrial |
| ELOVL4 | ELOVL fatty acid elongase 4 |
| TGM6 | Transglutaminase 6 |
| NOP56 | NOP56 ribonucleoprotein |
| ELOVL5 | ELOVL fatty acid elongase 5 |
| CCDC88C | Coiled-coil domain containing 88C |
| KCNA1 | Potassium voltage-gated channel, shaker-related |
| | subfamily, member 1 |
| CACNB4 | Calcium channel, voltage-dependent, beta 4 subunit |
| SLC1A3 | EAAT1 (excitatory amino acid transporter type 1) |
| FXN | Frataxin |
| TTPA | Tocopherol (alpha) transfer protein (ataxia (Friedreich- |
| | like) with vitamin E deficiency) |
| C10orf2 | chromosome 10 open reading frame 2 |
| APTX | Aprataxin |
| SETX | Senataxin |
| SYNE1 | Spectrin repeat containing, nuclear envelope 1 (nesprin 1) |
| ADCK3 | Atypical kinase ADCK3, mitochondrial |
| TDP1 | Tyrosyl-DNA phosphodiesterase 1 |
| SIL1 | SIL1 homolog, endoplasmic reticulum chaperone |
| POLG | Polymerase (DNA directed), gamma |
| ATM | Ataxia telangiectasia mutated |
| MRE11A | MRE11 meiotic recombination 11 homolog A |
| SACS | Sacsin |
| PHYH | Phytanoyl-CoA 2-hydroxylase |
| PEX7 | Peroxisomal biogenesis factor 7 |
| RNF216 | Ring finger protein 216 |

Any one of the above listed genes may be targeted in replacement gene therapy, wherein the gene of interest is a functional version of the deficient or mutated gene.

Alternatively, the above listed genes may be used as target for gene editing. Gene editing is used to correct the sequence of a mutated gene or modify the expression or regulation of a deficient/abnormal gene so that a functional gene is expressed in muscle cells. In such cases, the gene of interest is chosen from those encoding therapeutic RNAs such as interfering RNAs, guide RNAs for genome editing and antisense RNAs capable of exon skipping, wherein the therapeutic RNAs target the preceding list of genes. Tools such as CRISPR/Cas9 may be used for that purpose.

Thus, by gene editing or gene replacement a correct version of this gene is provided in target cells of affected patients, in particular muscle cells of affected patients, this may contribute to effective therapies against this disease.

In some embodiments, the target gene for gene therapy (additive gene therapy or gene editing) is a gene responsible for a muscular disorder, or a neuromuscular disorder, as disclosed herein.

Disease that can treated by gene therapy using the AAV vector according to the invention include in particular: Muscular dystrophies, Congenital muscular dystrophies, Congenital myopathies, Distal myopathies, Other myopathies, Myotonic syndromes, Ion Channel muscle diseases, Malignant hyperthermia, Metabolic myopathies, Hereditary Cardiomyopathies, Congenital myasthenic syndromes, Myastenia, Spinal muscular atrophies (SMAs) and Motor Neuron diseases, Hereditary paraplegia, Hereditary ataxia, Hereditary motor and sensory neuropathies and other neuromuscular disorders; the disease can be treating by targeting the gene associated with said diseases as listed in the Tables above.

These diseases may be classified in different groups: (i) Myopathies include hereditary cardiomyopathies, metabolic myopathies, other myopathies, distal myopathies, muscular dystrophies and congenital myopathies; muscular dystrophies include Duchenne muscular dystrophies; congenital myopathies include myotubular myopathy and centronuclear myopathies; other myopathies include oculopharyngeal muscular dystrophy (or OPMD; PABPN1 gene) (ii) Spinal muscular atrophies (SMAs) and motor neuron diseases include amyotrophic lateral sclerosis (ALS), progressive bulbar palsy (PBP), pseudobulbar palsy, progressive muscular atrophy (PMA), primary lateral sclerosis (PLS), spinal muscular atrophy (SMA) and monomelic atrophy (MMA), as well as some rarer variants resembling ALS; (iii) Myotonic syndrome includes myotonic dystrophy type 1 also known as Steinert's disease (DMPK gene) and type 2 (CNBP gene); (iv) Congenital myasthenic syndromes and Myastenia; (v) Hereditary motor and sensory neuropathies; (vi) Hereditary paraplegia and Hereditary ataxia. Inflammatory Myopathies (e.g. polymyositis dermatomyositis, inclusion-body myositis); diseases of neuromuscular junction (e.g. myasthenia gravis, Lambert-Eaton (myasthenic) syndrome, congenital myasthenic syndromes); diseases of peripheral nerve (e.g. Charcot-Marie-Tooth disease, Friedreich's ataxia, Dejerine-Sottas disease); metabolic diseases of muscle (e.g. phosphorylase deficiency (McArdle disease), acid maltase deficiency (Pompe disease), phosphofructokinase deficiency (Tarui disease), debrancher enzyme deficiency (Cori or Forbes disease), mitochondrial myopathy, carnitine deficiency, carnitine palmityl transferase phosphoglycerate kinase deficiency, deficiency (PGK1), phosphoglycerate mutase deficiency, lactate dehydrogenase deficiency, myoadenylate deaminase deficiency); myopathies due to endocrine abnormalities (e.g. hyperthyroid myopathy, hypothyroid myopathy), and other myopathies (e.g. myotonia congenital, paramyotonia congenital, central core disease, nemaline myopathy, myotubular myopathy, periodic paralysis).

Examples of neuromuscular genetic disorders that can be treated using an AAV vector according to the disclosure are listed below:
Dystrophinopathies are a spectrum of X-linked muscle diseases caused by pathogenic variants in DMD gene, which encodes the protein dystrophin. Dystrophinopathies comprises Duchenne muscular dystrophy (DMD), Becker muscular dystrophy (BMD) and DMD-associated dilated cardiomyopathy.
The Limb-girdle muscular dystrophies (LGMDs) are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported (LGMD1A to LGMD1G; LGMD2A to LGMD2W). Calpainopathy (LGMD2A) is caused by mutation of the gene CAPN3 with more than 450 pathogenic variants described. Contributing genes to LGMD phenotype include: anoctamin 5 (ANO5), blood vessel epicardial substance (BVES), calpain 3 (CAPN3), caveolin 3 (CAV3), CDP-L-ribitol pyrophosphorylase A (CRPPA), dystroglycan 1 (DAG1), desmin (DES), DnaJ heat shock protein family (Hsp40) homolog, subfamily B, member 6 (DNAJB6), dysferlin (DYSF), fukutin related protein (FKRP), fukutin (FKT), GDP-mannose pyrophosphorylase B (heterogeneous nuclear ribonucleoprotein D like (HNRNPDL), LIM zinc finger domain containing 2 (LIMS2), lain A:C (LMNA), myotilin (MYOT), plectin (PLEC), protein O-glucosyltransferase 1 (PLOGLUT1), protein O-linked mannose N-acetylglucosaminyltransferase 1 (beta 1,2-) (POMGNT1), protein O-mannose kinase (POMK), protein O-mannosyltransferase 1 (POMT1), protein O-mannosyltransferase 2 (POMT2), sarcoglycan alpha (SGCA), sarcoglycan beta (SGCB), sarcoglycan delta (SGCD), sarcoglycan gamma (SGCG), titin-cap (TCAP), transportin 3 (TNPO3), torsin 1A interacting protein (TOR1AIP1), trafficking protein particle complex 11 (TRAPPC11), tripartite motif containing 32 (TRIM 32) and titin (TTN). Major contributing genes to LGMD phenotype include CAPN3, DYSF, FKRP and ANO5 (Babi Ramesh Reddy Nallamilli et al., Annals of Clinical and Translational Neurology, 2018, 5, 1574-1587.
The Emery-Dreifuss Muscular Dystrophy (EDMD) caused by defects in one of the gene including the EMD gene (coding for emerin), the FHL1 gene and the LMNA gene (encoding lamin A and C).
Nesprin-1 and Nesprin-2 related muscular dystrophy caused by defects in the SYNE1 and SYNE2 gene, respectively; LUMA related muscular dystrophy caused by defects in the TMEM43 gene; LAP1B related muscular dystrophy caused by defects in the TOR1AIP1 gene.
Facio-scapulo-humeral muscular dystrophy, type 1 (FSHD1A), such as associated with defect in the DUX4 gene (contraction of the D4Z4 macrosatellite repeat in the subtelomeric region of chromosome 4q35) or the FRG1 gene; Facio-scapulo-humeral muscular dystrophy, type 2 (FSHD1B) caused by defects in the SMCHD1 gene.
Dysferlin is involved in neurological disorders including multiple sclerosis (Hochmeister et al., J. Neuropathol. Exp. Neurol., 2006 September; 65 (9): 855-65); Alzheimer (Galvin et al., Acta Neuropathol., 2006 December; 112 (6): 665-71 and choreic movement (Takahashi T, et al., Mov. Disord., 2006 September; 21 (9): 1513-5).
Spinal muscular atrophy is a genetic disorder caused by mutations in the Survival Motor Neuron 1 (SMN1) gene which is characterized by weakness and wasting (atrophy) in muscles used for movement. Mutations in ASAH1 gene lead to SMA-PME (spinal muscular atrophy with progressive myoclonic epilepsy).
Centronuclear myopathies include X-linked myoatubular myopathy (MTM1), autosomal and recessive dominant centronuclear myopathies (DNM2, BIN1, etc) as disclosed in the Tables above. X-linked myotubular myopathy is a genetic disorder caused by mutations in the myotubularin (MTM1) gene which affects muscles used for movement (skeletal muscles) and occurs almost exclusively in males. This condition is characterized by muscle weakness (myopathy) and decreased muscle tone (hypotonia).
Titinopathies are genetic disorders caused by mutations in the Titin (TTN) gene. Both dominant and recessive TTN mutations have been reported to cause a wide spectrum of cardiac and skeletal muscle diseases. Dominant titinopathies include hereditary myopathy with early respiratory failure (HMERF) caused by mutations in exon 344, and late-onset tibial muscular dystrophy (TMD). Recessive titinopathies include limb-girdle muscular dystrophy 2J, young- or early-adult-onset distal titinopathy, Emery-Dreifuss-like myopathy without cardiomyopathy, and congenital myopathy with or without heart disease.
Glycogen storage disease is a group of inherited metabolic disorders involving enzymes responsible for the synthesis and degradation of glycogen: GSDI (von Gierke's disease), GSDII (Pompe disease), GSDIII (Cori disease), GSDIV, GSDV, GSDVI, GSDVII, GSDVIII or lethal congenital glycogen storage disease of the heart:
Pompe disease (GSDII) is a genetic disorder caused by mutations in the acid alpha-glucosidase (GAA) gene. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.
Glycogen storage disease III (GSDIII or Cori disease) is an autosomal recessive metabolic disorder caused by homozygous or compound heterozygous mutation in the Amylo-Alpha-1, 6-Glucosidase, 4-Alpha-Glucanotransferase (AGL) gene which encodes the glycogen debrancher enzyme and associated with an accumulation of abnormal glycogen with short outer chains. Clinically, patients with GSD III present in infancy or early childhood with hepatomegaly, hypoglycemia, and growth retardation. Muscle weakness in those with IIIa is minimal in childhood but can become more severe in adults; some patients develop cardiomyopathy.
Genome-wide association studies identified the BIN1 locus as a leading modulator of genetic risk in Alzheimer's disease (AD) (Voskobiynyk et al., eLife doi: 10.7554/eLife.57354; Jul. 13, 2020). Hereditary spastic paraplegias (HSPs) are a group of rare, inherited, neurological diseases characterized by broad clinical and genetic heterogeneity. Lower-limb spasticity with first motoneuron involvement is the core symptom of all HSPs. The genes responsible for HSPs include at least 79 SPG genes. Mutations in SPG7 and SPAST are common causes of hereditary spastic paraplegia (HSP) (Review in Lallemant-Dudek P. et al. Fac. Rev., 2021 March 10; 10:27).
MECP2 (methylCpG binding protein 2) appears to be essential for the normal function of nerve cells. It is an important reader of DNA methylation. Its methyl-CpG-binding (MBD) domain recognizes and binds 5-mC regions. MECP2 gene is X-linked and subject to X inactivation. MECP2 gene mutations are the cause of most cases of Rett Syndrome a progressive neurologic developmental disorder and one of the most common causes of cognitive disability in females. At least 53 disease-causing mutations in this gene have been discovered.

Other diseases that can treated by gene therapy using the AAV vector according to the invention include lysosomal storage diseases (LSD), such as mucopolysaccharidosis type I to VII (MPSI-VII), Sandhoff disease and Tay-Sachs and metabolic diseases such as Maple syrup disease (MSUD), Methylmalonic academia (MMA), glycogenosis type I and III (GSDI and III], Niemann-Pick disease (NPC), Canavan disease, and Phenylketonuria (PKU).

In some particular embodiments, the target gene for gene therapy (additive gene therapy or gene editing) is a gene responsible for a neuromuscular disease selected from the group comprising: Duchenne muscular dystrophy (DMD gene); Limb-girdle muscular dystrophies (LGMDs) (CAPN3, DYSF, FKRP, ANO5, DNAJB6 genes and others such as SGCA, SGCB, SGCG); Spinal muscular atrophy (SMN1, ASAH1 genes); Myotubular myopathy (MTM1 gene); Glycogen storage diseases, in particular Pompe disease (GAA gene); Glycogen storage disease III (AGL gene).

In a more particular embodiment, the disease is a glycogen storage such as GSDI, GSDII, GSDIII, GSDIV, GSDV, GSDVI, GSDVII, GSDVIII or lethal congenital glycogen storage disease of the heart; more particularly GSDII or GSDIII; even more particularly GSDII. In a further particular embodiment, the disorder is Pompe disease and the therapeutic transgene is a gene encoding an acid alpha-glucosidase (GAA) or a variant thereof. Such variants of GAA are in particular disclosed in applications PCT/2017/072942, PCT/EP2017/072945, PCT/EP2017/072944 and WO 2019/154939. In a particular embodiment, the disorder is infantile-onset Pompe disease (IOPD) or late onset Pompe disease (LOPD). Preferably, the disorder is IOPD.

In the various embodiments of the present invention, the pharmaceutical composition comprises a therapeutically effective amount of AAV vector or cell. In the context of the invention a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies. The term “effective dose” or “effective dosage” is defined as an amount sufficient to achieve, or at least partially achieve, the desired effect. The therapeutic dose of vector induces an immune tolerance to the transgene.

The effective dose is determined and adjusted depending on factors such as the composition used, the route of administration, the physical characteristics of the individual under consideration such as sex, age and weight, concurrent medication, and other factors, that those skilled in the medical arts will recognize. The effective dose can be determined by standard clinical techniques. In addition, in vivo and/or in vitro assays may optionally be employed to help predict optimal dosage ranges.

In the various embodiments of the present invention, the pharmaceutical composition comprises a pharmaceutically acceptable carrier and/or vehicle.

A “pharmaceutically acceptable carrier” refers to a vehicle in which the therapeutic is administered and that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The pharmaceutical composition may also comprise an additional therapeutic agent, in particular an agent useful for the treatment of a muscle disease including neuromuscular disease according to the present disclosure.

The AAV vector, cell or pharmaceutical composition of the invention may be used in combination with other biologically active agents, wherein the combined use is by simultaneous, separate or sequential administration.

Another aspect of the invention relates to the AAV vector cell, pharmaceutical composition according to the present disclosure as a medicament, in particular for use in the treatment of a muscle disease including neuromuscular disease according to the present disclosure.

The invention provides also a method for treating a muscle disease including neuromuscular disease according to the present disclosure, comprising: administering to a patient a therapeutically effective amount of the pharmaceutical composition as described above, comprising at least an active agent selected from an AAV vector or a cell of the invention, and a pharmaceutically acceptable carrier.

A further aspect of the invention relates to the use of an AAV vector, cell according to the present disclosure in the manufacture of a medicament for the treatment of a muscle disease including neuromuscular disease according to the present disclosure.

Another aspect of the invention relates to the use of a AAV vector particle or a cell of the present disclosure for the treatment of a muscle disease including neuromuscular disease according to the present disclosure.

A further aspect of the invention relates to a pharmaceutical composition for treatment of a muscle disease including neuromuscular disease according to the present disclosure, comprising an AAV vector or a cell of the present disclosure as an active component.

A further aspect of the invention relates to a pharmaceutical comprising an AAV vector particle or a cell of the present disclosure for treating a muscle disease including neuromuscular disease according to the present disclosure.

As used herein, the term “patient” or “individual” includes human and other mammalian subjects that receive either prophylactic or therapeutic treatment. Said mammal may be an infant or adult subject, such as human infant or human adult. Preferably, a patient or individual according to the invention is a human.

Treatment”, or “treating” as used herein, is defined as the application or administration of a therapeutic agent or combination of therapeutic agents to a patient, or application or administration of said therapeutic agents to an isolated tissue or cell line from a patient, who has a disease with the purpose to cure, heal, alleviate, relieve, alter, remedy, ameliorate, improve or affect the disease, or any symptom of the disease. In particular, the terms “treat’ or treatment” refers to reducing or alleviating at least one adverse clinical symptom associated with the disease.

The term “treatment” or “treating” is also used herein in the context of administering the therapeutic agents prophylactically.

The pharmaceutical composition of the present invention is generally administered according to known procedures, at dosages and for periods of time effective to induce a therapeutic effect in the patient. The pharmaceutical composition may be administered by any convenient route, such as in a non-limiting manner by infusion or bolus injection, by absorption through epithelial or mucocutaneous linings (e.g., oral mucosa, rectal and intestinal mucosa, etc.). The administration can be systemic, local or systemic combined with local; systemic includes parenteral and oral, and local includes local and loco-regional. Systemic administration is preferably parenteral such as subcutaneous (SC), intramuscular (IM), intravascular such as intravenous (IV) or intraarterial; intraperitoneal (IP); intradermal (ID), epidural or else. The parenteral administration is advantageously by injection or perfusion. In some preferred embodiments, the administration is parenteral, preferably intravascular such as intravenous (IV) or intraarterial.

The various embodiments of the present disclosure can be combined with each other and the present disclosure encompasses the various combinations of embodiments of the present disclosure.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1. A. Schematic representation of the engineered AAV-MT capsid. B. Design of the LiMP-hGAA cassette. The transgene expression cassette, flanked by the two AAV2 ITRs, is composed of an ApoE enhancer and the tandem hAAT-SpC5-12 promoter (LiMP), a SV40 intron, a codon-optimized human GAA cDNA (hGAAco) and the bGH polyadenylation signal.

FIG. 2. Combination of AAV-MT with LiMP leads to increased hGAA expression and better glycogen clearance in adult Gaa^{-/-} mice at short-term. A. Experimental design. 4-month-old Gaa^{-/-} mice received a single injection of AAV9-SpC5-12-hGAAco, AAV9-LiMP-hGAAco or AAV-MT-LiMP-hGAAco at 3×10¹² vector genomes (vg)/kg (n=4 per group). PBS-injected Gaa^{+/+} (n=4) and Gaa^{-/-} (n=4) mice were used as controls. Red symbols indicate the timing of blood collection in all cohorts. B. Analysis of GAA activity and C. glycogen content in heart and muscle one month after vector injection. D. GAA activity in liver. E Circulating anti-hGAA IgG measured overtime. Data shown as mean±SD. Statistical analysis; B-E: One-way ANOVA with Tuckey post hoc; * and ^{#}p<0.05, ** and ^{##}p<0.01, *** and ⁺⁺⁺p<0.001, ****, ⁺⁺⁺⁺ and ^{####}p<0.0001.

FIG. 3. 4-month-old Gaa^{-/-} mice received a single injection of AAV9-SpC5-12-hGAAco, AAV9-LiMP-hGAAco or AAV-MT-LiMP-hGAAco at 3×10¹² vg/kg (n=4 per group). PBS-injected Gaa^{+/+} (n=4) and Gaa^{-/-} (n=4) mice were used as controls. A-B. Analysis of vector genome copy number (VGCN) in muscles (A) and liver (B) at sacrifice. Data shown as mean±SD. Statistical analysis; A-B: One-way ANOVA with Tuckey post hoc; *p<0.05, **p<0.01, ***p<0.001, **** and ****p<0.0001.

FIG. 4. Specific muscle-targeting with AAV-MT results in complete glycogen clearance in skeletal muscle in adult Pompe mice. A. Experimental design. 4-month-old Gaa^{-/-} mice received a single injection of AAV9-LiMP-hGAAco or AAV-MT-LiMP-hGAAco at 1×10¹² or 3×10¹² vg/kg (n=4 per group). PBS-injected Gaa^{+/+} (n=4) and Gaa^{-/-} (n=4) mice were used as controls. Red symbols indicate the timing of blood collection in all cohorts. B. Analysis of GAA activity in muscles three months after vector injection. C-D. Western blot analysis of hGAA in (C) quadriceps and (D) soleus. The quantification of hGAA protein band is plotted on the right. E. Analysis of glycogen content in muscles three months after vector injection. Data shown as mean±SD. Statistical analysis; B-E: One-way ANOVA with Tuckey post hoc; * and ⁺p<0.05, **, ⁺⁺ and ^{##}p<0.01, *** and ^{###}p<0.001, ****, ⁺⁺⁺⁺ and ^{####}p<0.0001.

FIG. 5. 4-month-old Gaa^{-/-} mice received a single injection of AAV9-LiMP-hGAAco or AAV-MT-LiMP-hGAAco at 1×10¹² or 3×10¹² vg/kg (n=4 per group). PBS-injected Gaa^{+/+} (n=4) and Gaa^{-/-} (n=4) mice were used as controls. A-C. GAA activity (A), glycogen content (B) and vector genome copy number (VGCN, C) measured in muscles three months after vector injection. D. Analysis of grip strength 3 months after treatment. E. Circulating anti-hGAA IgG measured 0.5- and 2-months post injection. Data shown as mean±SD. Statistical analysis; A-E: One-way ANOVA with Tuckey post hoc; *p<0.05, **, ++ and ^{##}p<0.01, *** and +++p<0.001, **** and ^{####}p<0.0001.

FIG. 6. Residual liver transgene expression with AAV-MT combined with LiMP reduces anti-hGAA humoral response in adult Pompe mice. Four-month-old Gaa^{-/-} mice were treated as described in FIG. 2. A-B. Analysis of vector genome copy number (VGCN) (A) and GAA activity (B) in liver at sacrifice. C-D. Western blot analysis of hGAA in (C) liver and (D) blood. The quantification of the hGAA protein band is plotted on the right. E. Circulating anti-hGAA IgG measured overtime. F. Circulating anti-hGAA IgG measured at 1- and 3-months post injection. Data shown as mean±SD. Statistical analysis; A-F: One-way ANOVA with Tuckey post hoc; * and ⁺p<0.05, ** and ⁺⁺p<0.01, ***p<0.001, **** and ⁺⁺⁺⁺p<0.0001.

### EXAMPLES

### 1. Materials and Methods

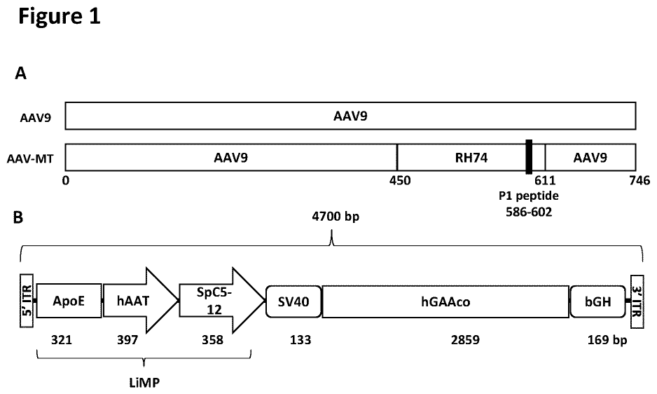

AAV vectors. AAV-MT-LiMP and AAV9-LiMP comprise a codon-optimized human GAA transgene (hGAAco) operably linked to the LiMP promoter, a SV40 intron and a bGH polyadenylation signal as previously disclosed in WO 2019/154939 (FIG. 1B). AAV-MT capsid is derived from hybrid AAV9.rh74 capsid and comprises the insertion of muscle-targeting peptide P1 flanked by 4 aa (GQSGRGDLGLSAQAA) in place of residues 587 to 592 of the capsid sequence as previously disclosed in WO 2020/200499; the AAV-MT capsid corresponds to SEQ ID NO: 54 and comprises the P1 peptide insertion between positions 586 (Q586) and 602 (1602) as shown in FIG. 1A.

AAV vector production. AAV-MT-LiMP and AAV9-LiMP vectors were produced by an adenovirus-free transient tri-transfection method of HEK 293 cells in suspension and purified by affinity chromatography. Titers of AAV vectors were determined using qPCR and all vector preparations used in the studies were quantified side by side.

Mouse model. Comparative efficacy studies were performed in male Gaa knockout mice (Gaa^{-/-}), purchased from The Jackson Laboratory (B6; 129-Gaa^{tm1Rabn/J}, stock number 004154, 6neo) and originally generated by Raben et al. (J Biol Chem, 1998. 273 (30): p. 19086-92). Littermate male mice were used, either affected (Gaa^{-/-}) or healthy (Gaa^{+/+}). The phenotype of males Gaa^{-/-} versus Gaa^{+/+} from the colonies has been reported in previous work (Puzzo, F., et al., Sci Transl Med, 2017. 9 (418)).

In vivo studies in mice. In vivo studies were performed in compliance with all relevant ethical regulations for animal testing and research. Notably, they were performed according to the French and European legislation on animal care and experimentation (2010/63/EU) and approved by Genethon's ethical committee. In all mouse studies, animals were randomly assigned to treatment groups. To minimize potential bias during functional assessments in mice, operators were blinded to the study design. Operators in charge of sample analysis were blinded to study design.

Experiments in adult mice. Treatments with AAV vectors were performed in male Gaa^{-/-} mice of four months of age, that received either intravenous injection of vehicle (PBS), AAV9-LiMP or AAV-MT-LiMP. Four animals per group were injected via tail vein infusion with 1×10¹² or 3×10¹² vg/kg of vector (in a volume of 200 μL). Age-matched and sex-matched Gaa^{+/+} littermates were used as healthy controls in the studies.

Experiment in neonate mice. Male Gaa^{-/-} mice from birth to two days of age were treated either with PBS, AAV9-LiMP or AAV-MT-LiMP vectors. Three animals per group were injected via tail vein infusion with 1×10¹³ or 3×10¹³ vg/kg of vector (in a volume of 20 μL). Age-matched and sex-matched Gaa^{+/+} littermates were used as healthy controls in the studies.

Blood GAA activity and anti-hGAA IgG measurement. Blood samples were collected by retro-orbital sampling into heparinized capillary tubes and mixed with 3.8% w/v sodium citrate, followed by plasma isolation. GAA activity measurement was performed as already described [20]. The concentration of anti-hGAA IgG antibodies in mouse plasma was determined by enzyme linked immunosorbent assay (ELISA) (Puzzo et al., precited).

Tissue GAA activity and Western blot analyses. Snap-frozen tissues were homogenized in UltraPure DNase/RNase-free water (Thermo Fisher Scientific, Waltham, MA) with FastPrep lysis tubes (MP Biomedicals, Ohio, USA), followed by centrifugation 20 min at 10,000×g to collect the supernatant. Protein content in lysates was quantified by BCA Protein Assay (Thermo Fisher Scientific). GAA activity measurement was performed as already described [20]. SDS-page electrophoresis was performed with NuPAGE 4-12% Bis-Tris protein gels (Life technologies, Carlsbad, CA). After transfer, the membrane was blocked with Odyssey buffer (Li-Cor Biosciences) and incubated with an anti-hGAA antibody (rabbit monoclonal, clone EPR4716 (2), Abcam) and anti-Vinculin (mouse monoclonal, clone V9131, Sigma-Aldrich). Membrane was then washed and incubated with the appropriate secondary antibody (LI-COR Biosciences) and visualized with the Odyssey imaging system (Li-Cor Biosciences). Densitometry analysis was conducted using Image Studio Lite (Li-Cor Biosciences) version 4.0. The quantification of the hGAA bands in mouse tissues was normalized using housekeeping Vinculin protein bands. Protein level was reported in units of arbitrary unity (AU).

Glycogen content measurement. Tissue homogenates samples were prepared as described for the analysis of GAA activity. Glycogen assay was performed as already described (Puzzo et al., precited).

Vector genome copy number. Vector genome copies in mice were determined by qPCR on total tissue DNA. Total DNA was extracted from tissues homogenates using NucleoMag Pathogen (Macherey-Nagel, Hoerdt, France) extraction method according to manufacturer's instructions. The number of vector copies per diploid genome was determined using primers to amplify the GAA transgene sequence (forward: 5′-AGATACGCCGGACATTGGACTG-3′ (SEQ ID NO: 57); reverse: 5′-GTTCAATCTGCTGGGCGTGC-3′ (SEQ ID NO: 58); probe: 5′-GTGTGGTCCTCTTGGGAGC-3′ (SEQ ID NO: 59)). The number of vector copies was normalized by the copies of the titin gene, which was used as an internal control for each sample (forward: 5′-AAAACGAGCAGTGACGTGAGC-3′ (SEQ ID NO: 60); reverse: 5′-TTCAGTCATGCTGCTAGCGC-3′ (SEQ ID NO: 61); probe, 5′-TGCACGGAAGCGTCTCGTCTCAGT-3′ (SEQ ID NO: 62)). Data were expressed as vector genome copies per diploid genome.

Histology and staining. Immediately after euthanasia, muscles were snap-frozen in isopentane (−160° C.) previously chilled in liquid nitrogen. Serial 8 mm cross-sections were cut in a Leica CM3050 S cryostat (Leica Biosystems, Wetzlar, Germany). To minimize sampling error, 2 or 3 sections of each specimen were obtained and stained with periodic acid-Schiff (PAS) according to standard procedures. Muscle images were acquired using an Axioscan slide scanner (ZEISS, Munich, Germany), using a plan-apochromat 10× magnitude 0.45 NA objective.

Grip Test. Muscle strength was assessed using a grip strength meter (Columbus Instruments, Columbus, OH) (Zhang, P., et al., Hum Gene Ther, 2012. 23 (5): p. 460-72). Briefly, mice were lifted by the tail to the same height of the grip strength meter grid. Mice were then moved horizontally until they were within reach. Four-limb grip was inspected visually to confirm the symmetry and the tight grip. Mice were then gently pulled away from the grid until the grasp was released. The Grip strength meter recorded the value. Three independent measures were performed for each mouse. Mean values of three independent measures expressed in Newton were reported.

Statistical analysis. All data shown in the present manuscript are expressed as mean±standard deviation (SD). GraphPad Prism 7.0 software (GraphPad Software, San Diego, CA) was used for statistical analyses. p-value <0.05 was considered significant. The number of sampled units (n), upon which statistics was reported is the single animal. Parametric tests were used for data having a normal distribution with α=0.05. One-way ANOVA with Tukey's post hoc correction was used for comparisons of one variable between more than two groups. All statistical tests were performed two-sided. The statistical analysis performed for each dataset is indicated in the figure legends.

### 2. Results

### AAV-MT Mediated Gene Transfer Results in Superior Clearance of Glycogen in Skeletal Muscle of Adult Gaa^{-/-} Mice.

Simultaneous expression of an engineered, hGAA (Δ8-hGAAco) in liver and muscle with an AAV9 under the transcriptional control of the LiMP has shown efficacy in Gaa^{-/-} mice (Colella, P., et al., Mol Ther Methods Clin Dev, 2019. 12: p. 85-101; WO 2019/154939). The muscle-targeting specificity achieved by grafting RGD-containing peptides was exploited to evaluate the contribution of muscle-specific hGAA expression by LiMP (El Andari, J., et al., Sci Adv, 2022. 8 (38): p. eabn4704; Tabebordbar, M., et al., Cell, 2021. 184 (19): p. 4919-4938 e22; Weinmann, J., et al., Nat Commun, 2020. 11 (1): p. 5432) in an AAV capsid with enhanced liver detargeting (WO 2019/193119). The inventors have cloned the RGDLGLS peptide (P1) between amino acids 586 and 602 of a new AAV capsid generated by the combination of AAV9 and AAV-RH74 (FIG. 1A) following a previously described method (Weinmann et al., precited). The resulting capsid, named AAV-MT (for Muscle Transduction, WO 2020/200499), was used to express the native form of codon-optimized (co), human GAA (hGAAco) (Puzzo et al., precited) under the transcriptional control of the LiMP (FIG. 1B).

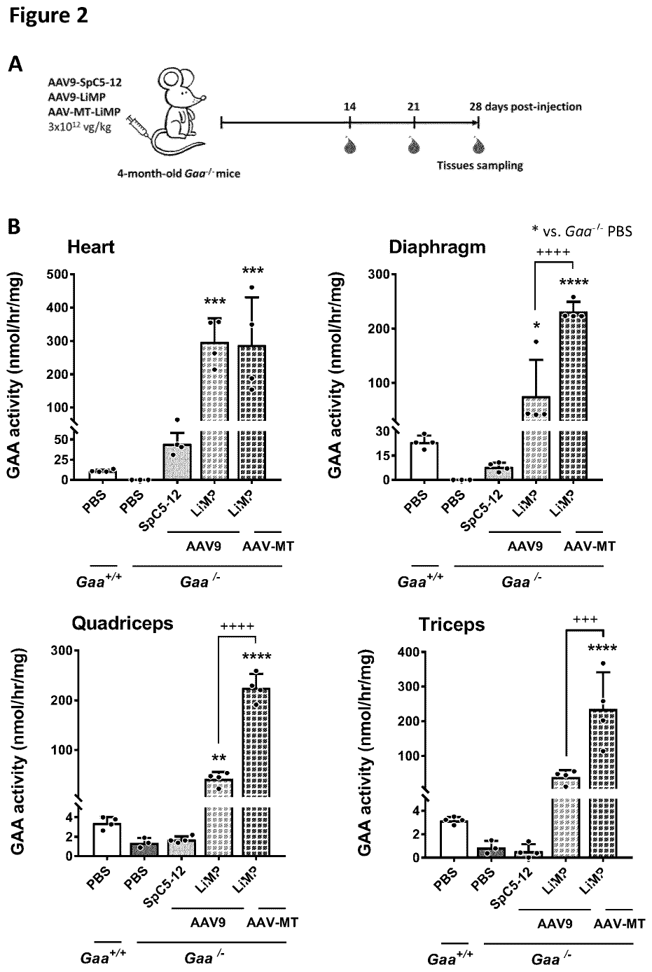

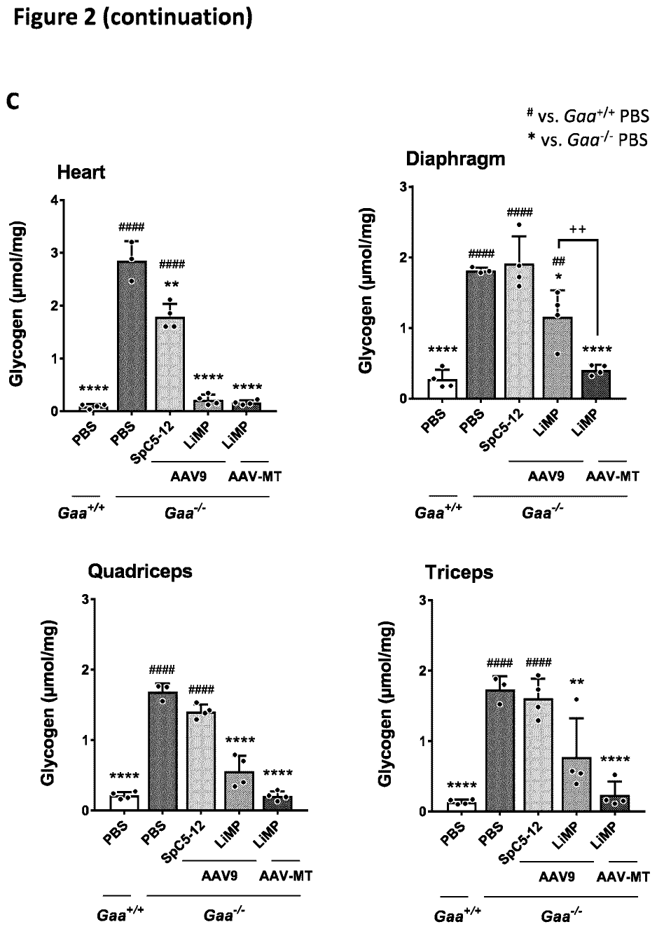

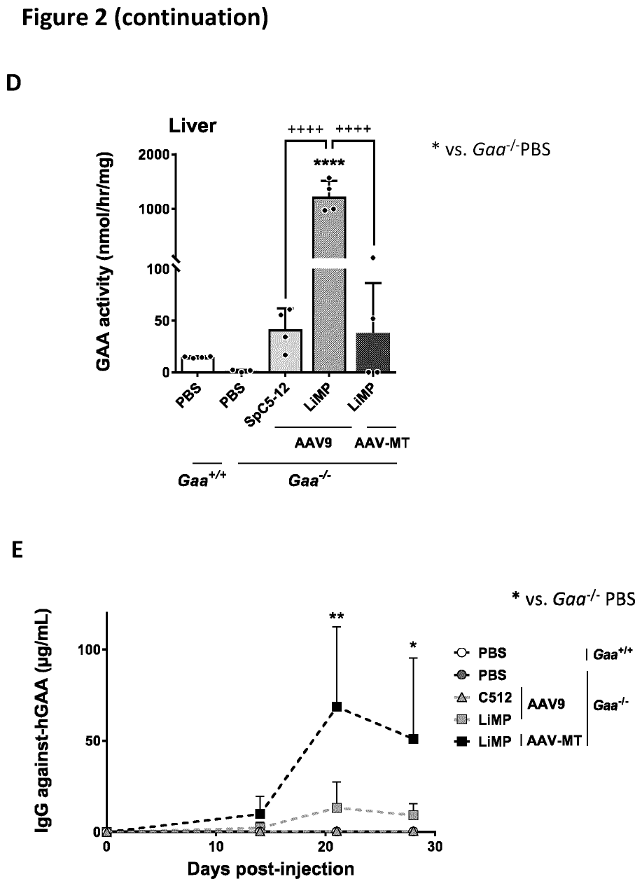

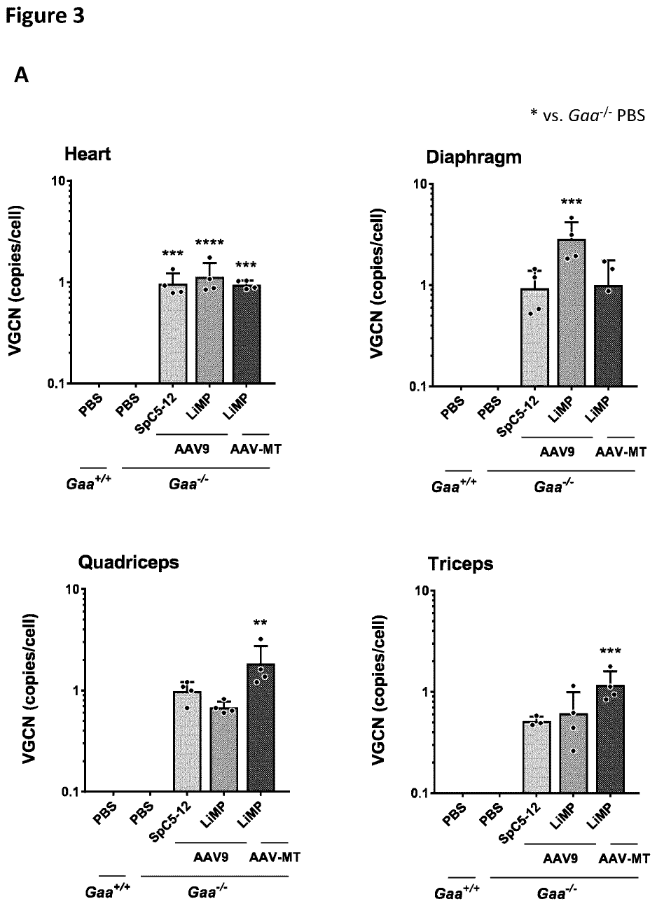

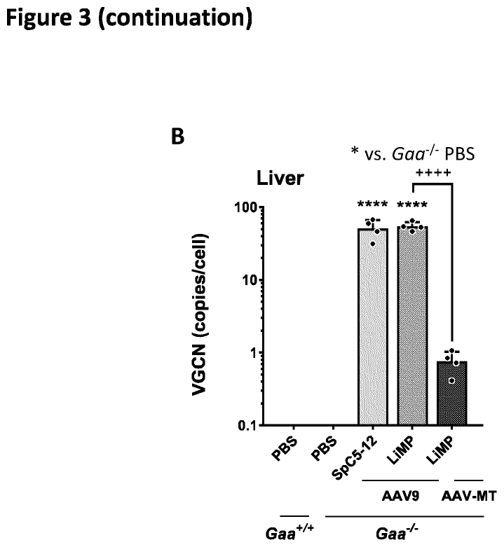

In a first experiment performed in four-month-old Gaa^{-/-} mice, the short-term efficacy of an AAV-MT vector expressing hGAAco under the control of the LiMP was compared to the efficacy obtained when the same transgene was expressed by AAV9 either using SpC5-12, a known muscle promoter, or LiMP (FIG. 2A). One month after injection of the three vectors at the dose of 3×10¹² vg/kg, in line with what was previously described (Colella, P., et al., precited), a marked improvement of GAA activity was observed in heart and in different muscle groups in mice treated with AAV9-LiMP, compared to mice treated with AAV9-SpC5-12 (FIG. 2B). When compared to AAV9, the combination of AAV-MT capsid with the LiMP, while resulting in similar heart transduction, led to a striking increase in skeletal muscle targeting, as shown by GAA activity (FIG. 2B) and vector genome copy number (VGCN) analysis (FIG. 3A). Increased GAA activity was invariably associated with improved glycogen clearance (FIG. 2C), thus supporting the potential of AAV-MT in muscle targeting and rescue of phenotype in Gaa^{-/-} mice. As expected, high hepatic GAA activity was observed in mice injected with AAV9-LiMP, whereas animals receiving AAV-MT with the same promoter showed low GAA activity, comparable to that measured in animals injected with AAV9-SpC5-12 (FIG. 2D). The extensive liver detargeting of the AAV-MT was also confirmed by the VGCN analysis in this tissue (FIG. 3B). Given the central role of the liver in establishing peripheral tolerance to antigens expressed in muscle (Franco, L. M., et al., Mol Ther, 2005. 12 (5): p. 876-84; Zhang, P., et al., Hum Gene Ther, 2012. 23 (5): p. 460-72; Poupiot, J., et al., Molecular Therapy-Methods & Clinical Development, 2019. 15: p. 83-100; Bartolo, L., et al., JCI insight, 2019. 4 (11)) the increased muscle expression and the decreased liver targeting, achieved by the combination of AAV-MT capsid and LiMP, resulted in increased levels of circulating anti-hGAA antibodies measured three and four weeks after vector injection (FIG. 2E). These data suggest that the use of AAV-MT combined with LiMP can improve glycogen clearance in Gaa^{-/-} mice while reducing the dose and detargeting the liver.

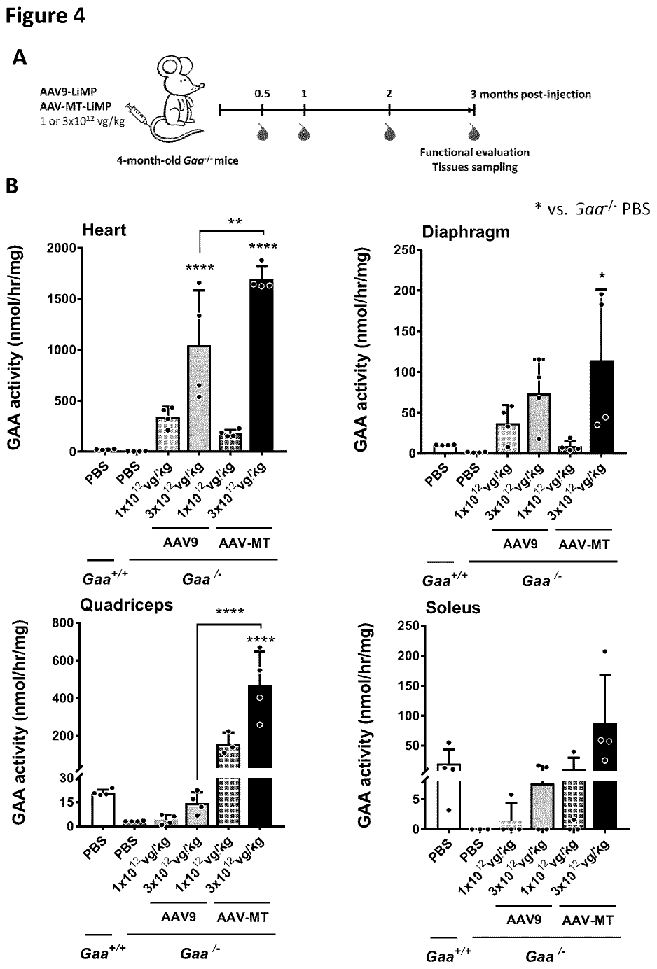

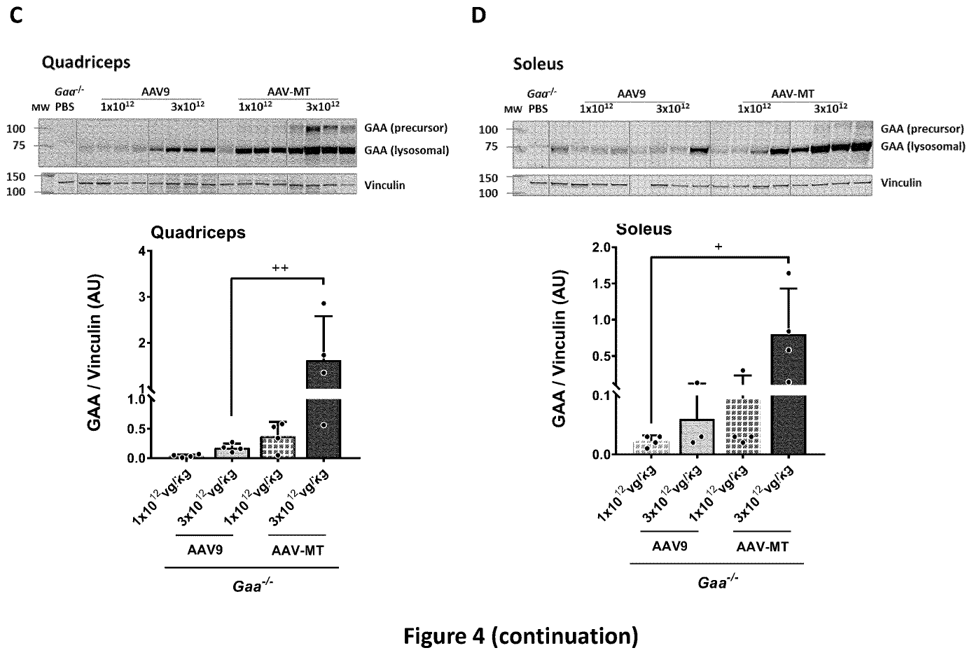

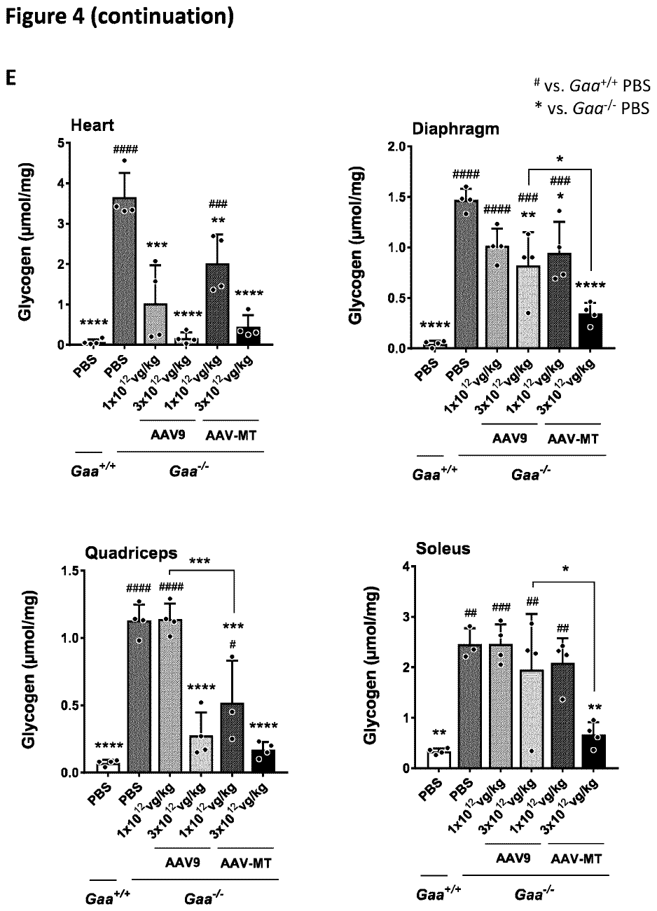

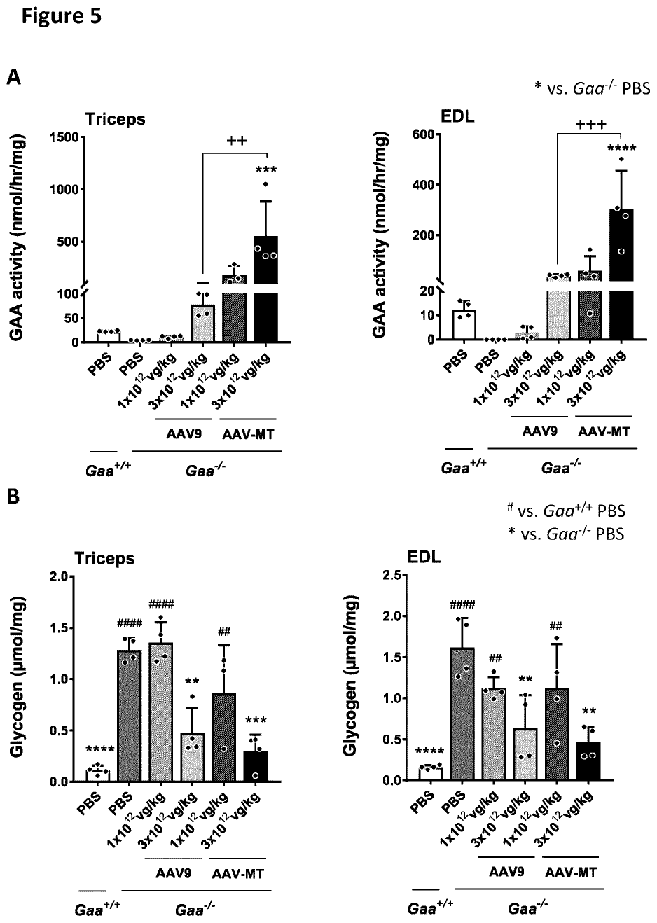

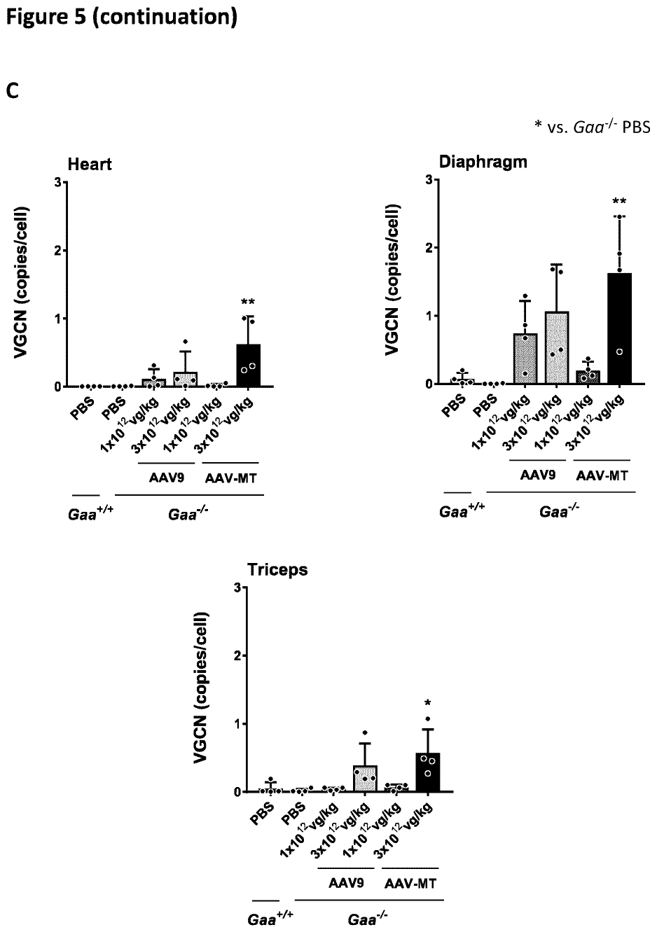

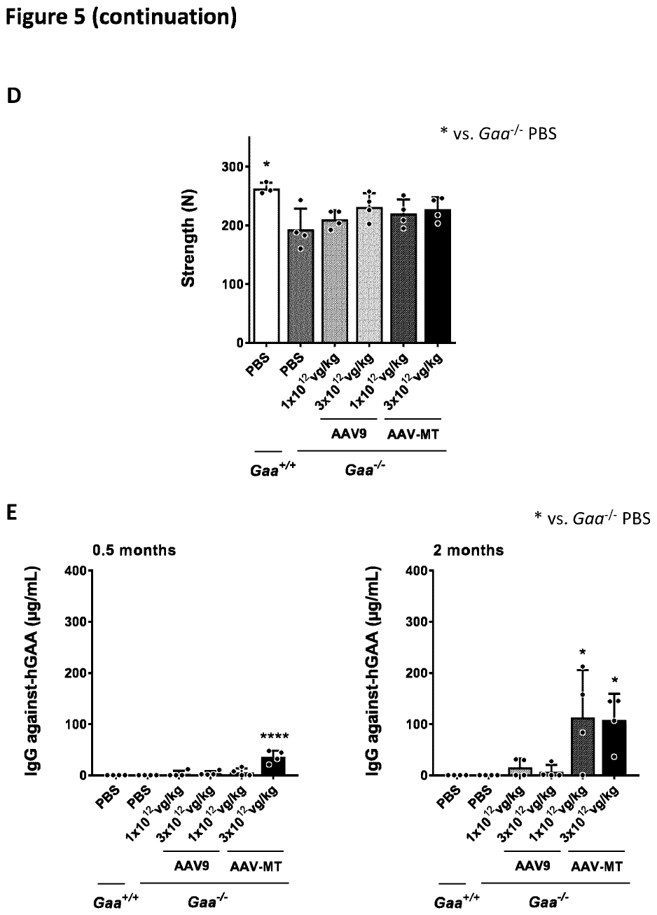

To confirm the increased muscle targeting of this novel AAV-MT capsid compared to AAV9, and to assess its therapeutic potential and immunogenicity when combined with the LiMP, the comparison was extended at two different doses. For this purpose, four-month-old Gaa^{-/-} mice were injected with the two vectors at 1×10¹² or 3×10¹² vg/kg and sacrificed three months later to compare vector efficacy (FIG. 4A). At the lower dose, while GAA activity was similar in heart and diaphragm regardless of the treatment received by Gaa^{-/-} mice, a tendency for higher GAA activity was observed in skeletal muscles of mice treated with AAV-MT vector (FIG. 4B and FIG. 5A). In the two groups treated at the higher vector dose, a significantly higher GAA activity was measured in cardiac and skeletal muscles of AAV-MT injected animals (FIG. 4B and FIG. 5A), also confirmed by GAA quantification by Western blot analysis in quadriceps and soleus (FIG. 4C, D). Consistently, increased GAA protein and activity was associated to better glycogen clearance in skeletal muscles (FIG. 4E). Treatment with AAV-MT-LiMP resulted in complete clearance of glycogen from heart and all the muscles tested at the dose of 3×10¹² vg/kg (FIG. 4E and FIG. 5B). Importantly, at this dose, AAV9-LiMP was not able to clear glycogen from diaphragm and soleus. At the lower dose, 1×10¹² vg/kg, AAV-MT showed a significantly better efficacy in glycogen clearance in quadriceps, with a tendency toward a better clearance in EDL and triceps (FIG. 4E and FIG. 5B). The higher muscle targeting of AAV-MT capsid was further confirmed by the tendency to achieve higher VGCNs measured in heart and triceps of Gaa^{-/-} mice injected with this vector when compared to AAV9 (FIG. 5C). Grip-strength assay performed at the end of the study, three months after AAV injection, showed a tendency for an increased muscle strength in all treated animal, regardless of the AAV vector, although none of the groups reached a statistical significance, possibly due to the low number of mice used in this analysis (FIG. 5D).

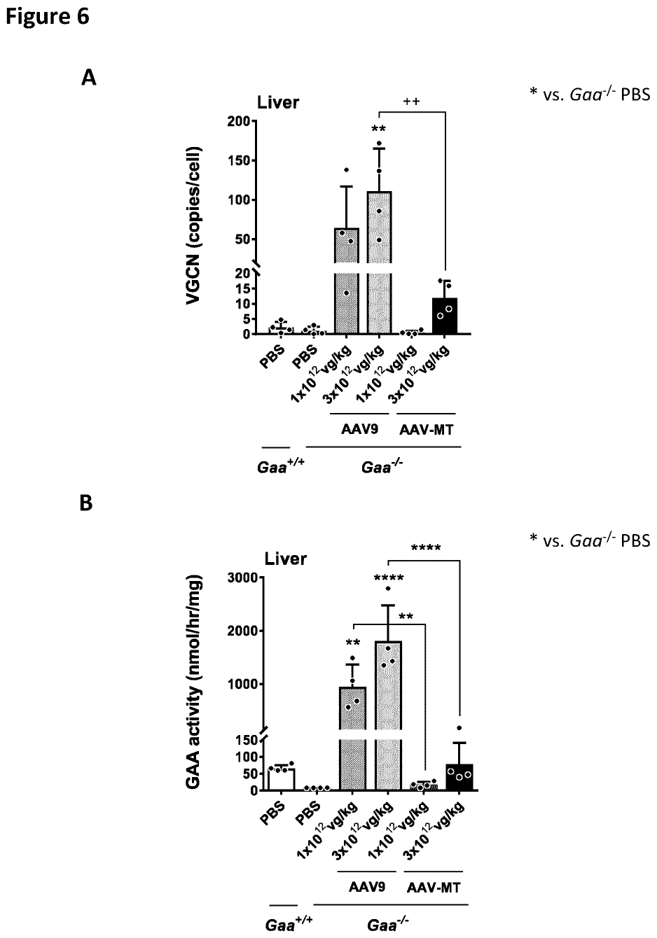

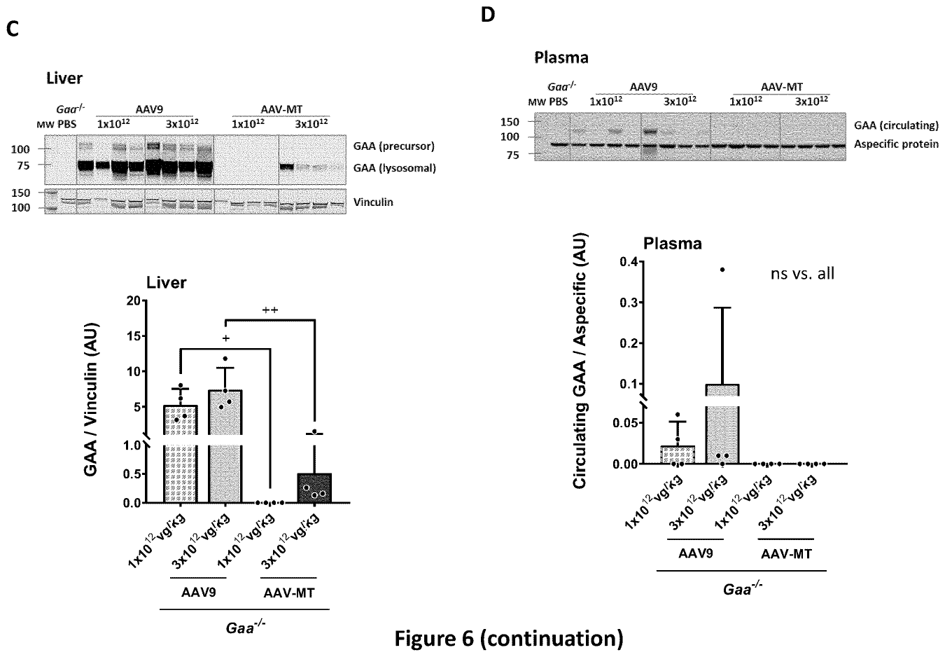

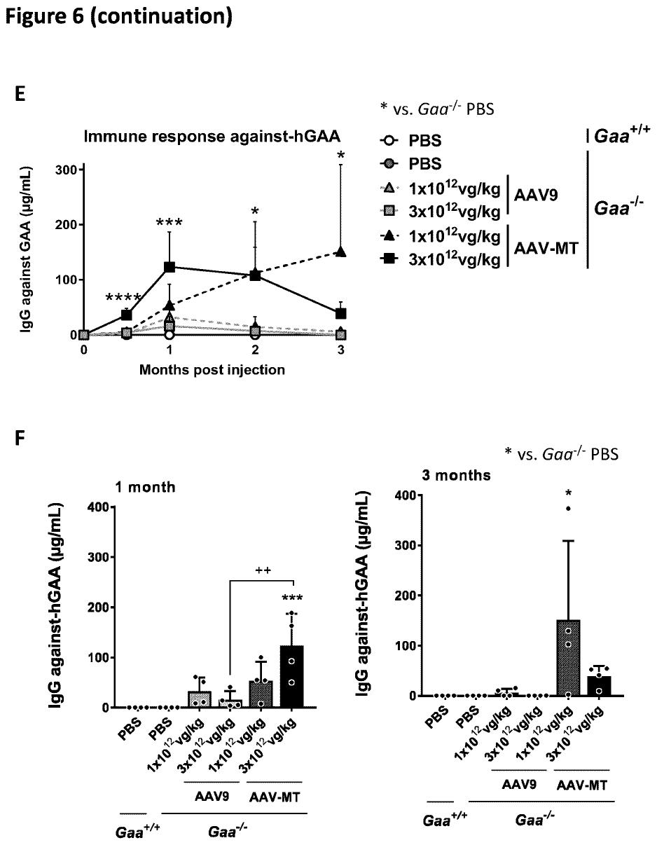

The insertion of the P1 peptide between amino acids 586 and 602 abolished the binding to heparan sulfate, central to liver targeting in rodents (Pulicherla, N., et al., Mol Ther, 2011. 19 (6): p. 1070-8), in an AAV capsid that showed already a pronounced liver de-targeting (WO 2019/193119). As expected, liver VGCN analysis showed robust targeting with AAV9 and reduced copies per cell when the new capsid was used, regardless of the dose (FIG. 6A). The combination of LiMP and AAV9 allowed for a robust expression of the transgene in liver, whereas with AAV-MT, due to the lower liver transduction, very low GAA activity levels were measured (FIG. 6B). Western blot analysis confirmed the activity results with a strong hGAA band in AAV9-LiMP-injected Gaa^{-/-} mice (FIG. 6C). Importantly, Gaa^{-/-} mice treated with AAV-MT-LiMP vector at the dose of 3×10¹² vg/kg showed a faint band consistent with the size of mature hGAA protein, thus indicating some residual liver expression in this treatment group (FIG. 6C). Circulating hGAA was relatively low across the different groups, possibly due to the use of the native form of hGAA (Puzzo et al., precited), although it was possible to measure circulating hGAA by Western blot in animals treated with AAV9-LiMP (FIG. 6D). These results confirm a strong, dose-dependent liver de-targeting by the AAV-MT.

AAV-mediated, hepatocyte-specific expression of a transgene induces a strong peripheral tolerance to transgenes expressed in muscle (Franco, L. M., et al., Mol Ther, 2005. 12 (5): p. 876-84; Zhang, P., et al., Hum Gene Ther, 2012. 23 (5): p. 460-72; Poupiot, J., et al., Molecular Therapy-Methods & Clinical Development, 2019. 15: p. 83-100; Bartolo, L., et al., JCI insight, 2019. 4 (11)). Consistently, in a previous work, it was demonstrated that combined expression of hGAA in liver and muscle with LiMP resulted in very low humoral immunity to hGAA in Gaa^{-/-} mice, compared to a specific-muscle promoter (Colella et al., precited). To understand how liver detargeting by AAV-MT impacts the humoral response against the protein, the anti-hGAA IgG levels were followed overtime through an ELISA test (FIG. 6E). Gaa^{-/-} mice treated with AAV9-LiMP showed a low and transient humoral immune response to hGAA that peaked at one-month post-injection and almost completely disappeared at the end of the study, three months post-injection (FIG. 6E, F and FIG. 5E). As expected, a dose-dependent increase in the level of anti-hGAA IgG was measured in AAV-MT treated mice at one month after injection (FIG. 6E, F and FIG. 5E). Importantly, in animals injected with the lower dose, of AAV-MT-LiMP vector the levels of circulating anti-hGAA IgG increased overtime, whereas at the higher dose, anti-hGAA IgG decreased dramatically, most probably due to the residual expression of hGAA in liver.

Taken together, these results indicate a dose advantage for efficient muscle correction in the use of a muscle-specific AAV capsid when combined with a LiMP. They also reinforce the concept of residual liver expression as beneficial in the control of immune responses to the transgene.

### Muscle-Specific Targeting Achieves Long-Term Efficacy and Functional Rescue in Neonate Gaa^{-/-} Mice.

Neonate Gaa^{-/-} mice were injected right after birth (post-natal day 0 to 2) with 1×10¹³ and 3×10¹³ vg/kg of AAV9 or AAV-MT vectors expressing hGAAco under the control of the LiMP. Six months after vector injection, mice were sacrificed to compare the efficacy of the two vectors (data not shown). As expected, injection of neonate mice resulted in AAV vector genome dilution overtime with low copies measured in liver at the end of the study, regardless the treatment (0.42±0.3 and 2.17±0.8 for AAV9; 0.15±0.2 and 0.46±0.3 for AAV-MT, respectively at 1×10¹³ vg/kg and 3×10¹³ vg/kg). Activity and expression of hGAA was observed in liver tissue in Gaa^{-/-} mice treated with AAV9-LiMP vector at both doses (data not shown). Very low hGAA expression was observed also in the liver of animals treated with the AAV-MT-LiMP vector at the higher dose (data not shown). The measurement of circulating hGAA by Western blot confirmed the data of liver expression, with a consistent hGAA bands detected in the blood of AAV9 treated Gaa^{-/-} mice and lower levels in the animals treated with AAV-MT at the dose of 3×10¹³ vg/kg (data not shown). GAA activity in heart was significantly higher in mice injected with AAV9 compared to AAV-MT (data not shown), and this correlated with higher VGCN measured in this tissue (data not shown). Despite the low GAA activity levels, complete correction of glycogen accumulation was observed in the hearts of all AAV-treated Gaa^{-/-} mice, starting from the lower dose (data not shown), confirming that low levels of GAA are needed to clear glycogen from that tissue (Costa-Verdera H., et al., Nat Commun, 2021. 12 (1): p. 6393). In skeletal muscle, increased GAA activity was measured in diaphragm, quadriceps, and triceps of Gaa^{-/-} mice injected with AAV-MT, that reached significance at the higher dose (data not shown). In line with these results, Western blot analysis showed enhanced hGAA expression in triceps of AAV-MT treated mice, at 3×10¹³ vg/kg (data not shown). Increased GAA activity resulted in better glycogen clearance in all muscle groups with a dose advantage for AAV-MT in quadriceps and triceps, and a tendency in diaphragm as measured by glycogen content (data not shown) and PAS staining (data not shown). Extensive glycogen clearance was also observed in soleus and EDL muscles. Importantly, at the lower dose, complete clearance of glycogen was observed in the soleus of Gaa^{-/-} mice treated with AAV-MT, whereas rodents treated with AAV9 had levels comparable to those treated with PBS (data not shown). In EDL, complete glycogen clearance was measured in AAV-treated animals, all doses and vectors confounded (data not shown). PAS staining performed on these muscles confirmed the dose advantage of the AAV-MT vector, with better glycogen clearance in soleus at the higher dose (data not shown).

Assessment of functional improvements was performed to explore potential correction of cardiomegaly and improvement of muscle strength in AAV-treated Gaa^{-/-} mice. Heart weight measurement showed a tendency to the increase in PBS-treated Gaa^{-/-} mice although it did not reach significance, possibly due to the low number of animals considered. However, AAV-treated animals showed in general lower heart weight, similar to PBS-treated Gaa^{+/+} mice (data not shown). In line with the complete clearance of glycogen in skeletal muscle, rescue of muscle strength was observed at four and six months after AAV-MT treatment at the higher dose (data not shown). Gaa^{-/-} mice treated with AAV9 vector at the lower dose were not rescued at both time points, thus confirming the dose advantage of muscle-specific targeting with AAV-MT vector.

Taken together, these data further support the use of muscle-specific targeting for the complete rescue of the Pompe phenotype in neonate Gaa^{-/-} mice at doses relevant for the clinical translation of the approach.

### DISCUSSION

Since 1998, year of the first report of AAV gene therapy for Pompe disease (Tsujino, S., et al., Human gene therapy, 1998. 9 (11): p. 1609-1616), a number of different approaches were reported to provide a curative treatment for this devastating disease (Ronzitti, G., et al., Ann Transl Med, 2019. 7 (13): p. 287). Muscle being the primary tissue affected by the lack of GAA activity, its targeting by gene therapy was considered since the very beginning for the treatment of the disease and one clinical trial is ongoing using this approach (NCT04174105). As an alternative, the use of the liver to secrete the hGAA protein in the circulation has been proposed (Sun, B., et al., Mol Ther, 2005. 11 (1): p. 57-65; Sun, B., et al., Mol Ther, 2006. 14 (6): p. 822-30). It was previously showed that liver expression of a secretable hGAA effectively reduced the dose of AAV vector needed to clear glycogen from muscle cells and allowed for a complete rescue of the muscle function at very low AAV doses (Puzzo, F., et al., Sci Transl Med, 2017. 9 (418); Cagin, U., et al., Molecular Therapy, 2020; Costa-Verdera, H., et al., Nat Commun, 2021. 12 (1): p. 6393). However, this approach is limited by liver growth during infancy and the resulting AAV vector dilution. On the other hand, muscle growth seems to have a lower effect on AAV genome dilution thus making muscle-directed gene therapy relevant for the treatment of classic infantile-onset Pompe disease (IOPD). One important limitation associated to muscle targeting with AAV vectors, is that the high doses necessary to achieve efficient transgene expression in muscle and the consequent liver overloading due to non-specific targeting are responsible for an important part of the severe adverse events reported in neuromuscular gene therapy (Nat Biotechnol, 2020.38 (8): p. 910).

Here, the inventors have demonstrated the potential of the use of an AAV capsid grafted with an RGD-containing peptide to transduce muscle tissues in vivo while de-targeting the liver in a mouse model of PD. The efficacy of the new capsid was compared side-by-side at multiple doses with a state-of-the-art approach for the correction of neonate mice recently developed in their laboratory (Colella et al., precited). The combination of AAV-MT and LiMP resulted in a two-log increase in hGAA expression and enhanced glycogen clearance in different muscle groups when compared to an AAV9 vector bearing a muscle-specific promoter, SpC5-12. Importantly, due to the peculiar biodistribution profile of the AAV-MT, an only residual liver expression of the transgene, comparable to that of SpC5-12, was observed despite the use of LiMP, a strong promoter in liver (Colella et al., precited). Extensive liver de-targeting with tremendously increased muscle targeting resulted in a robust humoral response to the transgene that may be detrimental. To confirm this hypothesis and to further evaluate the humoral response against hGAA after muscle-specific gene transfer, a multi-dose comparison was performed in adult Gaa^{-/-} mice. The use of an AAV9 vector in combination with LiMP was very efficient, the result of simultaneous muscle and liver targeting. Interestingly, despite the specific muscle targeting achieved by AAV-MT-LiMP, this vector demonstrated a clear dose-advantage when compared to AAV9-LiMP. Intriguingly, although AAV-MT-treated animals showed greater humoral response to hGAA than AAV9-treated Pompe mice, residual liver expression of hGAA, observed with the higher dose, resulted in reduced humoral response at the end of the study, three months after treatment. Most importantly, despite this apparently higher humoral response at one month which decline progressively to a reduced level at 3 months onwards, specific muscle targeting achieved by AAV-MT-LiMP, demonstrated a clear dose-advantage in muscle correction when compared to AAV9-LiMP.

Finally, to demonstrate the full potential of muscle-specific targeting in PD, an experiment was performed in neonate animals, where the liver transduction achieved with AAV9 is diluted overtime, thus allowing a direct comparison of muscle transduction with little to none circulating hGAA. Despite the fast liver growth, and due to the high doses needed to target the muscle in neonate animals, liver expression (corresponding to 50% of the activity measured in Gaa^{+/+} animals) was reported in AAV9-injected animals, whereas it was almost absent in AAV-MT treated animals, in particular at the lower dose. Despite this bias toward AAV9, the data in neonate Gaa^{-/-} mice indicate, as observed in adult animals, a dose advantage for the AAV-MT-LiMP vector. Indeed, six months after treatment, the results showed an almost complete clearance of glycogen in multiple muscle groups, starting from the lower dose of AAV-MT. Moreover, a functional rescue of muscle strength at both four and six months after vector injection in Gaa^{-/-} animals treated with 3×10¹³ vg/kg was observed.

One important open question in the use of AAV gene therapy for PD is the development of anti-hGAA antibodies in IOPD patients when they are CRIM−. Although it was demonstrated that even low levels of hGAA liver expression can reduce the humoral response to the transgene, the robustness of the induction of peripheral tolerance after liver gene transfer in humans is still questioned. However, protocols for tolerance induction are being developed for IOPD patients (Desai, A. K., et al., Ann Transl Med, 2019. 7 (13): p. 285) and, in combination with muscle-directed gene therapy, they may be able to provide an alternative treatment to ERT in this patients' population.

Noteworthy, the treatment in neonate Gaa^{-/-} mice required doses of vectors almost 10 times higher than the treatment of adult mice to achieve complete correction of the muscle phenotype six months after vector injection. Although this could be related primarily to a potential difference in efficacy of gene transfer in neonates compared to adult, one intriguing hypothesis is that during growth, the muscle mass increase may somehow dilute the vector although with a transgene loss less spectacular than that reported after liver growth (Nakai, H., et al., J Virol, 2001. 75 (15): p. 6969-76; Wang, L., et al., Molecular Therapy, 2011. 19 (11): p. 2012-2020; Cunningham, S.C., et al., Molecular Therapy, 2008. 16 (6): p. 1081-1088; Bortolussi, G., et al., FASEB J, 2012. 26 (3): p. 1052-63; Mingozzi, F. and K. A., Nat Rev Genet, 2011. 12 (5): p. 341-55). If this growth-dependent vector dilution in muscle is confirmed and given the formation of high titer anti-AAV antibodies after the injection of AAV vectors, strategies may be needed to be able to cope with vector re-administration. Recently, the use of IdeS (Leborgne, C., et al., Nature Medicine, 2020: p. 1-6; Elmore, Z. C., et al., JCI Insight, 2020. 5 (19); Ros-Ganan, I., et al., Clin Transl Immunology, 2022. 11 (2): p. e1375) or AAV vector-specific plasmapheresis (Bertin, B., et al., Scientific reports, 2020. 10 (1): p. 1-11; Orlowski, A., et al., Mol Ther Methods Clin Dev, 2020. 16: p. 192-203) were proposed by different groups as a clinically-relevant approaches to reduce circulating anti-AAV antibodies and allow for vector re-administration.

To conclude, AAV vectors with enhanced muscle targeting and improved liver de-targeting represents an alternative to the existing gene therapy approaches for IOPD and other muscle disorders.

### Sequences Disclosed in the Present Application

SEQ ID NO: 1: spC5.12 promoter

| | |
|---|---|
| caccgeggtg gcggccgtcc gccctcggca ccatcctcac gacacccaaa tatggcgacg | 60 |
| ggtgaggaat ggtggggagt tatttttaga gcggtgagga aggtgggcag gcagcaggtg | 120 |
| ttggcgctct aaaaataact cccgggagtt atttttagag cggaggaatg gtggacaccc | 180 |
| aaatatggcg acggttcctc acccgtcgcc atatttgggt gtccgccctc ggccggggcc | 240 |
| gcattcctgg gggccgggcg gtgctcccgc ccgcctegat aaaaggetcc ggggccggcg | 300 |
| gcggcccacg agctacccgg aggagcggga ggcgccaagc tctagaacta gtggatct | 358 |

SEQ ID NO: 2: E-Syn promoter

| | |
|---|---|
| cactacgggt ctaggctgcc catgtaagga ggcaaggcct ggggacaccc gagatgcctg | 60 |
| gttataatta accccaacac ctgctgcccc ccccccccca acacctgctg cctgagectg | 120 |
| agcggttacc ccaccccggt gcctgggtct taggctctgt acaccatgga ggagaagctc | 180 |
| gotctaaaaa taaccotgtc cctggtggcg cgccgagctc caccgcggtg goggccgtcc | 240 |
| gccctcggca ccatcctcac gacacccaaa tatggcgacg ggtgaggaat ggtggggagt | 300 |
| tatttttaga gcggtgagga aggtgggcag gcagcaggtg ttggcgctct aaaaataact | 360 |
| cccgggagtt atttttagag cggaggaatg gtggacaccc aaatatggcc caaatatggc | 420 |
| gacggttcct cacccgtcgc catatttggg tgtccgccct cggccggggc cgcattcctg | 480 |
| ggggccgggc ggtgctcccg cccgcctcga taaaaggctc cggggccggc ggcggcccac | 540 |
| gagctacccg gaggageggg aggcgccaag ctctagaact agtggatccc ccgggctgca | 600 |
| ggaattcgat at | 612 |

SEQ ID NO: 3: ApoE enhancer

| | |
|---|---|
| aggctcagag gcacacagga gtttctggge tcaccctgcc cccttccaac ccctcagttc | 60 |
| ccatcctcca gcagctgttt gtgtgctgcc tctgaagtcc acactgaaca aacttcagcc | 120 |
| tactcatgtc cctaaaatgg gcaaacattg caagcagcaa acagcaaaca cacagccctc | 180 |
| cctgcctgct gaccttggag ctggggcaga ggtcagagac ctctctgggc ccatgccacc | 240 |
| tccaacatcc actcgacccc ttggaatttc ggtggagagg agcagaggtt gtcctggcgt | 300 |
| ggtttaggta gtgtgagagg g | 321 |

SEQ ID NO: 4: hAAT promoter

| | |
|---|---|
| gatcttgcta ccagtggaac agccactaag gattctgcag tgagagcaga gggccagcta | 60 |
| agtggtactc toccagagac tgtctgacte acgccacccc ctccaccttg gacacaggac | 120 |
| gctgtggttt ctgagccagg tacaatgact cctttcggta agtgcagtgg aagctgtaca | 180 |
| ctgcccagge aaagcgtccg ggcagcgtag gegggcgact cagateccag ccagtggact | 240 |
| tagcccctgt ttgctcctcc gataactggg gtgaccttgg ttaatattca ccagcagcct | 300 |
| cccccgttgc ccctctggat ccactgctta aatacggacg aggacagggc cctgtctcct | 360 |
| cagcttcagg caccaccact gacctgggac agtgaat | 397 |

SEQ ID NO: 5: Enh.C5.12
ApoE enhancer (positions 1-321); linker (positions 322-325); spC5.12
promoter (positions 326-683)

| | |
|---|---|
| aggctcagag gcacacagga gtttctgggc tcaccctgcc cccttccaac ccctcagttc | 60 |
| ccatcctcca gcagctgttt gtgtgctgcc tctgaagtcc acactgaaca aacttcagcc | 120 |
| tactcatgtc cctaaaatgg gcaaacattg caagcagcaa acagcaaaca cacagccctc | 180 |
| cctgcctgct gaccttggag ctggggcaga ggtcagagac ctctctgggc ccatgccacc | 240 |
| tccaacatcc actcgacccc ttggaatttc ggtggagagg agcagaggtt gtcctggcgt | 300 |
| ggtttaggta gtgtgagagg ggtaccaccg cggtggcggc cgtccgccct cggcaccatc | 360 |
| ctcacgacac ccaaatatgg cgacgggtga ggaatggtgg ggagttattt ttagagcggt | 420 |
| gaggaaggtg ggcaggcage aggtgttggc gctctaaaaa taactcccgg gagttatttt | 480 |
| tagagcggag gaatggtgga cacccaaata tggcgacggt tectcacccg tcgccatatt | 540 |
| tgggtgtccg ccctcggccg gggccgcatt cctgggggcc gggcggtgct cccgcccgcc | 600 |
| tcgataaaag gctccggggc cggcggcggc ccacgagcta cccggaggag cgggaggcgc | 660 |
| caagctctag aactagtgga tct | |

SEQ ID NO: 6: LiMP
ApoE enhancer (positions 1-321); linker (positions 322-330); hAAT promoter
(positions 331-727); linker (positions 728-763); spC5.12 promoter
(positions 764-1121)

| | |
|---|---|
| aggctcagag gcacacagga gtttctgggc tcaccctgcc cccttccaac ccctcagttc | 60 |
| ccatcctcca gcagctgttt gtgtgctgcc tctgaagtcc acactgaaca aacttcagcc | 120 |
| tactcatgtc cctaaaatgg gcaaacattg caagcagcaa acagcaaaca cacagccctc | 180 |
| cctgcctgct gaccttggag ctggggcaga ggtcagagac ctctctgggc ccatgccacc | 240 |
| tccaacatcc actcgacccc ttggaatttc ggtggagagg agcagaggtt gtcctggcgt | 300 |
| ggtttaggta gtgtgagagg ggtacccggg gatcttgcta ccagtggaac agccactaag | 360 |
| gattctgcag tgagagcaga gggccagcta agtggtactc toccagagac tgtctgactc | 420 |
| acgccacccc ctccaccttg gacacaggac gctgtggttt ctgagccagg tacaatgact | 480 |
| cctttcggta agtgcagtgg aagctgtaca ctgcccagge aaagcgtccg ggcagcgtag | 540 |
| gcgggcgact cagatcccag ccagtggact tagcccctgt ttgctcctcc gataactggg | 600 |
| gtgaccttgg ttaatattca ccagcagcct cccccgttgc ccctctggat ccactgctta | 660 |
| aatacggacg aggacagggc cctgtctcct cagcttcagg caccaccact gacctgggac | 720 |
| agtgaataga tcctgagaac ttcagggtga gtctatggga ccccaccgcg gtggcggccg | 780 |
| tccgccctcg gcaccatect cacgacaccc aaatatggcg acgggtgagg aatggtgggg | 840 |
| agttattttt agagcggtga ggaaggtggg caggcagcag gtgttggcgc tctaaaaata | 900 |
| actcccggga gttattttta gagcggagga atggtggaca cccaaatatg gcgacggttc | 960 |
| ctcacccgtc gccatatttg ggtgtccgcc ctcggccggg gccgcattcc tgggggccgg | 1020 |
| gcggtgctcc cgcccgccte gataaaaggc tccggggccg gcggcggccc acgagctacc | 1080 |
| cggaggagcg ggaggcgcca agctctagaa ctagtggatc t | 1121 |

SEQ ID NO: 7: P1
RGDLGLS
SEQ ID NO: 8
LRGDGLS
SEQ ID NO: 9
LGRGDLS
SEQ ID NO: 10
LGLRGDS
SEQ ID NO: 11
LGLSRGD
SEQ ID NO: 12
RGDMSRE
SEQ ID NO: 13: P2
CDCRGDCFC
SEQ ID NO: 14: P3
RGDAVGV
SEQ ID NO: 15: Kera2
PRGDLAP
SEQ ID NO: 16
RGDVAAK
SEQ ID NO: 17
RGDMINT
SEQ ID NO: 18
RGDLNDS
SEQ ID NO: 19
RGDTMNY
SEQ ID NO: 20: MyoAAV 1A
RGDLTTP
SEQ ID NO: 21: MyoAAV 1B
RGDLNQY
SEQ ID NO: 22: MyoAAV 1C
RGDLSTP
SEQ ID NO: 23: MyoAAV 1D
RGDQLYH
SEQ ID NO: 24: MyoAAV 1E
RGDTMSK
SEQ ID NO: 25: MyoAAV 1F
RGDATEL
SEQ ID NO: 26: MyoAAV 2A
GPGRGDQTTL
SEQ ID NO: 27: MyoAAV 2B
AEGRGDQYTR
SEQ ID NO: 28: MyoAAV 2C
ATGRGDLGQA
SEQ ID NO: 29: MyoAAV 2D
AVARGDQGLI
SEQ ID NO: 30: MyoAAV 2E
NISRGDQGYQ
SEQ ID NO: 31: MyoAAV 2F
APARGDQGSQ
SEQ ID NO: 32: MyoAAV 2G
AVSRGDRMEF
SEQ ID NO: 33: MyoAAV 2H
SPSRGDQGRT
SEQ ID NO: 34: MyoAAV 3A
RGDYVGL
SEQ ID NO: 35: MyoAAV 3B
RGDYSGL
SEQ ID NO: 36: MyoAAV 3C
RGDYSSV
SEQ ID NO: 37: MyoAAV 3D
RGDYREL
SEQ ID NO: 38: MyoAAV 3E
RGDHGV
SEQ ID NO: 39: MyoAAV 3F
RGDHASW
SEQ ID NO: 40: MyoAAV 4A
SNSRGDYNSL
SEQ ID NO: 41: MyoAAV 4B
STVRGDYTS
SEQ ID NO: 42: MyoAAV 4C
QERRGDYTSM
SEQ ID NO: 43: MyoAAV 4D
ASTRGDHGV
SEQ ID NO: 44: MyoAAV 4E
ENRRGDENNT
SEQ ID NO: 45
GQSG
SEQ ID NO: 46
AQAA
SEQ ID NO: 47
GQSGRGDLGLSAQAA
SEQ ID NO: 48: AAV8
MAADGYLPDWLEDNLSEGIREWWALKPGAPKPKANQQKQDDGRGLVLPGYKYLGPFNGLD
KGEPVNAADAAALEHDKAYDQQLQAGDNPYLRYNHADAEFQERLQEDTSFGGNLGRAVFQ
AKKRVLEPLGLVEEGAKTAPGKKRPVEPSPQRSPDSSTGIGKKGQQPARKRLNFGQTGDS
ESVPDPQPLGEPPAAPSGVGPNTMAAGGGAPMADNNEGADGVGSSSGNWHCDSTWLGDRV
ITTSTRTWALPTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSEYQLPYVLGSA
HQGCLPPFPADVFMIPQYGYLTLNNGSQAVGRSSFYCLEYFPSQMLRTGNNFQFTYTFED
VPFHSSYAHSQSLDRLMNPLIDQYLYYLSRTQTTGGTANTQTLGFSQGGPNTMANQAKNW
LPGPCYRQQRVSTTTGQNNNSNFAWTAGTKYHLNGRNSLANPGIAMATHKDDEERFFPSN
GILIFGKQNAARDNADYSDVMLTSEEEIKTTNPVATEEYGIVADNLQQQNTAPQIGTVNS
QGALPGMVWQNRDVYLQGPIWAKIPHTDGNFHPSPLMGGFGLKHPPPQILIKNTPVPADP
PTTFNQSKLNSFITQYSTGQVSVEIEWELQKENSKRWNPEIQYTSNYYKSTSVDFAVNTE
GVYSEPRPIGTRYLTRNL
SEQ ID NO: 49: AAV9
MAADGYLPDWLEDNLSEGIREWWALKPGAPQPKANQQHQDNARGLVLPGYKYLGPGNGLD
KGEPVNAADAAALEHDKAYDQQLKAGDNPYLKYNHADAEFQERLKEDTSFGGNLGRAVFQ
AKKRLLEPLGLVEEAAKTAPGKKRPVEQSPQEPDSSAGIGKSGAQPAKKRLNFGQTGDTE
SVPDPQPIGEPPAAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAH
EGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHKEGEDRFFPLSGS
LIFGKQGTGRDNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIKNTPVPADPPT
AFNKDKLNSFITQYSTGQVSVEIEWELQKENSKRWNPEIQYTSNYYKSNNVEFAVNTEGV
YSEPRPIGTRYLTRNL
SEQ ID NO: 50: AAVrh74
MAADGYLPDWLEDNLSEGIREWWDLKPGAPKPKANQQKQDNGRGLVLPGYKYLGPFNGLD
KGEPVNAADAAALEHDKAYDQQLQAGDNPYLRYNHADAEFQERLQEDTSFGGNLGRAVEQ
AKKRVLEPLGLVESPVKTAPGKKRPVEPSPQRSPDSSTGIGKKGQQPAKKRLNFGQTGDS
ESVPDPQPIGEPPAGPSGLGSGTMAAGGGAPMADNNEGADGVGSSSGNWHCDSTWLGDRV
ITTSTRTWALPTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSEYQLPYVLGSA
HQGCLPPFPADVFMIPQYGYLTLNNGSQAVGRSSFYCLEYFPSQMLRTGNNFEFSYNFED
VPFHSSYAHSQSLDRLMNPLIDQYLYYLSRTQSTGGTAGTQQLLFSQAGPNNMSAQAKNW
LPGPCYRQQRVSTTLSQNNNSNFAWTGATKYHLNGRDSLVNPGVAMATHKDDEERFFPSS
GVLMFGKQGAGKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQILIKNTPVPADP
PTTFNQAKLASFITQYSTGQVSVEIEWELQKENSKRWNPEIQYTSNYYKSTNVDFAVNTE
GTYSEPRPIGTRYLTRNL
SEQ ID NO: 51: AAV9.rh74
MAADGYLPDWLEDNLSEGIREWWALKPGAPQPKANQQHQDNARGLVLPGYKYLGPGNGLD
KGEPVNAADAAALEHDKAYDQQLKAGDNPYLKYNHADAEFQERLKEDTSFGGNLGRAVEQ
AKKRLLEPLGLVEEAAKTAPGKKRPVEQSPQEPDSSAGIGKSGAQPAKKRLNFGQTGDTE
SVPDPQPIGEPPAAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAH
EGCLPPFPADVFMIPQYGYLTLNDGSQAVGRSSFYCLEYFPSQMLRTGNNFQFSYEFENV
PFHSSYAHSQSLDRLMNPLIDQYLYYLSRTQSTGGTAGTQQLLFSQAGPNNMSAQAKNWL
PGPCYRQQRVSTTLSQNNNSNFAWTGATKYHLNGRDSLVNPGVAMATHKDDEERFFPSSG
VLMFGKQGAGKDNVDYSSVMLTSEEEIKTTNPVATEQYGVVADNLQQQNAAPIVGAVNSQ
GALPGMVWQNRDVYLQGPIWAKIPHTDGNFHPSPLMGGFGMKHPPPQILIKNTPVPADPP
TAFNKDKLNSFITQYSTGQVSVEIEWELQKENSKRWNPEIQYTSNYYKSNNVEFAVNTEG
VYSEPRPIGTRYLTRNL
SEQ ID NO: 52: AAV9P1
MAADGYLPDWLEDNLSEGIREWWALKPGAPQPKANQQHQDNARGLVLPGYKYLGPGNGLD
KGEPVNAADAAALEHDKAYDQQLKAGDNPYLKYNHADAEFQERLKEDTSFGGNLGRAVFQ
AKKRLLEPLGLVEEAAKTAPGKKRPVEQSPQEPDSSAGIGKSGAQPAKKRLNFGQTGDTE
SVPDPQPIGEPPAAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAH
EGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHKEGEDRFFPLSGS
LIFGKQGTGRDNVDADKVMITNEEEIKTTNPVATESYGQVATNHQGQSGRGDLGLSAQAA
QTGWVQNQGILPGMVWQDRDVYLQGPIWAKIPHTDGNFHPSPLMGGFGMKHPPPQILIKN
TPVPADPPTAFNKDKLNSFITQYSTGQVSVEIEWELQKENSKRWNPEIQYTSNYYKSNNV
EFAVNTEGVYSEPRPIGTRYLTRNLEFAVNTEGVYSEPRPIGTRYLTRNL
SEQ ID NO: 53: AAV9. rh74-HB-P1
MAADGYLPDWLEDNLSEGIREWWALKPGAPQPKANQQHQDNARGLVLPGYKYLGPGNGLD
KGEPVNAADAAALEHDKAYDQQLKAGDNPYLKYNHADAEFQERLKEDTSFGGNLGRAVFQ
AKKRLLEPLGLVEEAAKTAPGKKRPVEQSPQEPDSSAGIGKSGAQPAKKRLNFGQTGDTE
SVPDPQPIGEPPAAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNRFHCHFSPRDWQR
LINNNWGFRPKRLNFKLENIQVKEVTDNNGVKTIANNLTSTVQVFTDSDYQLPYVLGSAH
EGCLPPFPADVFMIPQYGYLTLNDGSQAVGRSSFYCLEYFPSQMLRTGNNFQFSYEFENV
PFHSSYAHSQSLDRLMNPLIDQYLYYLSRTQSTGGTAGTQQLLESQAGPNNMSAQAKNWL
PGPCYRQQRVSTTLSQNNNSNFAWTGATKYHLNGRDSLVNPGVAMATHKDDEERFFPSSG
VLMFGKQGAGKDNVDYSSVMLTSEEEIKTTNPVATEQYGVVADNLQQQNAAARGDLGLSS
GAAPIVGAVNSQGALPGMVWQNRDVYLQGPIWAKIPHTDGNFHPSPLMGGFGMKHPPPQI
LIKNTPVPADPPTAFNKDKLNSFITQYSTGQVSVEIEWELQKENSKRWNPEIQYTSNYYK
SNNVEFAVNTEGVYSEPRPIGTRYLTRNL
SEQ ID NO: 54: AAV-MT
MAADGYLPDWLEDNLSEGIREWWALKPGAPQPKANQQHQDNARGLVLPGYKYLGPGNGLD
KGEPVNAADAAALEHDKAYDQQLKAGDNPYLKYNHADAEFQERLKEDTSFGGNLGRAVEQ
AKKRLLEPLGLVEEAAKTAPGKKRPVEQSPQEPDSSAGIGKSGAQPAKKRLNFGQTGDTE
SVPDPQPIGEPPAAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAH
EGCLPPFPADVFMIPQYGYLTLNDGSQAVGRSSFYCLEYFPSQMLRTGNNFQFSYEFENV
PFHSSYAHSQSLDRLMNPLIDQYLYYLSRTQSTGGTAGTQQLLFSQAGPNNMSAQAKNWL
PGPCYRQQRVSTTLSQNNNSNFAWTGATKYHLNGRDSLVNPGVAMATHKDDEERFFPSSG
VLMFGKQGAGKDNVDYSSVMLTSEEEIKTTNPVATEQYGVVADNLQGQSGRGDLGLSAQA
AIVGAVNSQGALPGMVWQNRDVYLQGPIWAKIPHTDGNFHPSPLMGGFGMKHPPPQILIK
NTPVPADPPTAFNKDKLNSFITQYSTGQVSVEIEWELQKENSKRWNPEIQYTSNYYKSNN
VEFAVNTEGVYSEPRPIGTRYLTRNL
SEQ ID NO: 55: AAVS1P1 or AAVMYO2
MAADGYLPDWLEDNLSEGIREWWALKPGAPQPKANQQHQDNARGLVLPGYKYLGPGNGLD
KGEPVNAADAAALEHDKAYDQQLKAGDNPYLRYNHADAEFQERLQEDTSFGGNLGRAVEQ
AKKRVLEPLGLVEEGAKTAPGKKRPVEPSPQRSPDSSTGIGKTGQQPAKKRLNFGQTGDT
ESVPDPQPIGEPPAAPAAVGPTTMASGGGAPMADNNEGADGVGNASGNWHCDSTWLGDRV
ITTSTRTWALPTYNNHLYKQISNGTSGGATNDNTYFGYSTPWGYFDFNRFHCHFSPRDWQ
RLINNNWGFRPKRLNFKLFNIQVKEVTDNNGVKTIANNLTSTVQVFTDSDYQLPYVLGSA
HEGCLPPFPADVFMIPQYGYLTLNDGSQAVGRSSFYCLEYFPSQMLRTGNNFQFSYEFEN
VPFHSSYAHSQSLDRLMNPLIDQYLYYLSKTINGSGQNQQTLKFSVAGPSNMAVQGRNYI
PGPSYRQQRVSTTVTQNNNSEFAWPGASSWALNGRNSLMNPGPAMASHKEGEDRFFPLSG
SLIFGKQGTGRDNVDADKVMITNEEEIKTTNPVATESYGQVATNHQGQSGRGDLGLSAQA
AQTGWVQNQGILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQYTSNYYKSNN
VEFAVNTEGVYSEPRPIGTRYLTRNL
SEQ ID NO: 56: AAVS10P1 or AAVMYO3
MAADGYLPDWLEDNLSEGIREWWALKPGAPKPKANQQKQDDGRGLVLPGYKYLGPFNGLD
KGEPVNAADAAALEHDKAYDQQLQAGDNPYLRYNHADAEFQERLQEDTSFGGNLGRAVEQ
AKKRLLEPLGLVEEAAKTAPGKKRPVEQSPQEPDSSAGIGKSGAQPAKKRLNFGQTGDTE
SVPDPQPIGEPPAAPSGVGSLTMASGGGAPVADNNEGADGVGNASGNWHCDSQWLGDRVI
TTSTRTWALPTYNNHLYKQISSASTGATNDNTYFGYSTPWGYFDFNRFHCHFSPRDWQRL
INNNWGFRPKRLNFKLFNIQVKEVTDNNGVKTIANNLTSTVQVFTDSDYQLPYVLGSAHE
GCLPPFPADVFMIPQYGYLTLNDGSQAVGRSSFYCLEYFPSQMLRTGNNFQFSYEFENVP
FHSSYAHSQSLDRLMNPLIDQYLYYLSKTINGSGQNQQTLKESVAGPSNMAVQGRNYIPG
PSYRQQRVSTTVTQNNNSEFAWPGASSWALNGRNSLMNPGPAMASHKEGEDRFFPLSGSL
IFGKQGTGRDNVDADKVMITNEEEIKTTNPVATESYGQVATNHQGQSGRGDLGLSAQAAQ
TGWVQNQGILPGMVWQDRDVYLQGPIWAKIPHTDGNFHPSPLMGGFGMKHPPPQILIKNT
PVPADPPTAFNKDKLNSFITQYSTGQVSVEIEWELQKENSKRWNPEIQYTSNYYKSNNVE
FAVNTEGVYSEPRPIGTRYLTRNL
SEQ ID NO: 57: GAA transgene forward primer
AGATACGCCGGACATTGGACTG
SEQ ID NO: 58: GAA transgene reverse primer
GTTCAATCTGCTGGGCGTGC
SEQ ID NO: 59: GAA transgene probe
GTGTGGTCCTCTTGGGAGC
SEQ ID NO: 60: titin gene forward primer
AAAACGAGCAGTGACGTGAGC
SEQ ID NO: 61: titin gene reverse primer
TTCAGTCATGCTGCTAGCGC
SEQ ID NO: 62: titin gene probe
TGCACGGAAGCGTCTCGTCTCAGT

1. An adeno-associated virus (AAV) vector comprising:
(i) a transgene of interest operably linked to a tandem promoter comprising a muscle-selective promoter fused to a liver-selective promoter; and
(ii) a peptide-modified AAV capsid protein which comprises the insertion of a muscle-targeting peptide comprising a RGD motif into the variable region VIII of AAV capsid protein sequence, wherein the AAV capsid is detargeted from the liver as the result of the peptide-insertion.
(iii)

2. The AAV vector according to claim 1, which induces immune tolerance to the transgene of interest.

3-16. (canceled)

17. The AAV vector according to claim 1, wherein the muscle-selective promoter is selected from the group consisting of: a spC5.12 promoter, a MHCK7 promoter, an E-syn promoter, a muscle creatine kinase myosin light chain (MLC) promoter, a myosin heavy chain (MHC) promoter, a desmin promoter, a cardiac troponin C promoter, a troponin I promoter, a myoD gene family promoter, an alpha actin promoter, an beta actin promoter, an gamma actin promoter, a muscle-selective promoter residing within intron 1 of the ocular form of Pitx3 and a CK6 promoter.

18. The AAV vector according to claim 1, wherein the muscle-selective promoter is spC5.12 promoter of SEQ ID NO: 1.

19. The AAV vector according to claim 1, wherein the liver-selective promoter is selected from the group consisting of: an alpha-1 antitrypsin (hAAT) promoter, a transthyretin promoter, an albumin promoter, a thyroxine-binding globulin (TBG) promoter and the LSP promoter.

20. The AAV vector according to claim 1, wherein the liver-selective promoter is the hAAT promoter of SEQ ID NO: 4.

21. The AAV vector according to claim 1, wherein the tandem promoter further comprises a muscle-selective enhancer and/or a liver-selective enhancer.

22. The AAV vector according to claim 1, wherein the tandem promoter further comprises the ApoE enhancer (ApoE) of SEQ ID NO: 3.

23. The AAV vector according to claim 1, wherein the tandem promoter comprises the combination of: (i) the ApoE enhancer and hAAT promoter and (ii) the spC5.12 promoter comprising SEQ ID NO: 5 or 6.

24. The AAV vector according to claim 1, wherein the gene of interest is a therapeutic gene.

25. The AAV vector according to claim 1, wherein the muscle-targeting peptide comprises a sequence selected from the group consisting of: SEQ ID NO: 7 to 44 and 47.

26. The AAV vector according to claim 1, wherein the peptide-modified AAV capsid protein is from AAV serotype selected from the group consisting of: AAV8, AAV9, AAVrh74, variants and hybrids thereof.

27. The AAV vector according to claim 1, wherein the peptide-modified AAV capsid protein is hybrid serotype AAV9.rh74, and AAV8, AAV9 or AAVrh74 hybrid serotypes comprising variable region(s) from AAV13 or hybrid AAV2/13.

28. The AAV vector according to claim 1, wherein the muscle-targeting peptide is inserted into a position of AAV capsid protein sequence selected from the group consisting of: position 585 or 590 in AAV8, position 588 or 589 in AAV9, position 589 in AAV9.rh74 and between positions 586 and 593 in AAV9.rh74.

29. The AAV vector according to claim 1, wherein the peptide-modified AAV capsid comprises a sequence having at least 95% identity with any one of SEQ ID NO: 52 to 56 which comprises a peptide of SEQ ID NO: 7.

30. The AAV vector according to claim 1, wherein the tandem promoter comprises SEQ ID NO: 6 and the peptide-modified AAV capsid comprises SEQ ID NO: 54.

31. A pharmaceutical composition comprising a therapeutically effective amount of AAV vector according to claim 1, or cell stably transduced by said AAV vector.

32. A method for gene therapy in a subject in need thereof comprising administering a therapeutically effective amount of the pharmaceutical composition of claim 31 to said subject.

33. The method of claim 32, for the treatment of muscle diseases; preferably selected from the group consisting of: Duchenne muscular dystrophy, Limb-girdle muscular dystrophies, Spinal muscular atrophy, Myotubular myopathy, Pompe disease and Glycogen storage disease III.

34. The method of claim 32, which targets a gene selected from the group comprising: DMD, CAPN3, DYSF, FKRP, DNAJB6, ANO5, SGCA, SGCB, SGCG, SMN1, ASAH, MTM1, GAA and AGL.
